SEQUENCE LISTING (8 sequences)

SEQ ID NO: 1 - DNA, 1228 bp, Sus scrofa
agagacgcagcagcgctccctttgcccacacccaccgctccctcgctcgcctctcctcccggagccagtccgcgccaccgcagccggcaggtggaccagctcaccaacgacaaggcccgcgtcgaggtggagcgcgacaatctggccgaggacatcatgcggctgcgggagaagttgcaggaggagacgcttcagagggaggaagccgagagcactttgcagtctttcagacaggatgtcgacaatgcttctttggcacgtcttgacctggagcgcaaagtggaatccttgcaagaagagattgcctttttgaagaaactgcacgatgaggaaatccaggagctgcaggctcagatccaggaacagcacgtccaaatcgatatggatgtttccaagcctgacctcacggctgccctgcgtgatgtccgccagcagtatgagagtgtggccgccaagaaccttcaggaggcggaggagtggtacaagtccaagtttgccgacctctccgaggctgctaaccgcaacaacgatgccctgcgccaggccaagcaggagtcaaatgagtaccggagacaggtgcagtctctcacctgtgaagtggatgcgctcaaggggaccaacgagtctctggaacgccagatgcgggaaatggaagagaactttgccgtggaagctgctaactaccaagacactattggccgcgtgcaggatgagattcagaacatgaaggaagagatggcccgtcaccttcgtgaataccaagacctgctgaatgtcaagatggctctcgacattgagattgccacctacaggaagctgctggaaggagaagagagcaggatttctctgcctcttccaaatttttcttccctgaacctgaggggttatcaatgaaacttcccagcatcacgatgatctggaataagaactgcatgtactctgtgcagcgaaatattaccagcaagaataaaaaagaaatccgtatcttcaagaaatagctttcaagtgcctttctgcagtttttcaggagcgcaagataggtttggaataggaataagctctagttcttaaaaaccaacactcgtaaaagatttaggaaaaaagtttacaacataatctagtttaccgaagaagccttgtgctagaatactttttaaaaagtatttttgaataccattaaaactgcttttttccatcaaatatccaaccaacttggttctgcttcaat

SEQ ID NO: 2 - protein, 403 aa, Sus scrofa
RDAAALPLPTPTAPSLASPPGASPRHRSRQVDQLTNDKARVEVERDNLAEDIMRLREKLQEETLQREEAESTLQSFRQDVDNASLARLDLERKVESLQEEIAFLKKLHDEEIQELQAQIQEQHVQIDMDVSKPDLTAALRDVRQQYESVAAKNLQEAEEWYKSKFADLSEAANRNNDALRQAKQESNEYRRQVQSLTCEVDALKGTNESLERQMREMEENFAVEAANYQDTIGRVQDEIQNMKEEMARHLREYQDLLNVKMALDIEIATYRKLLEGEESRISLPLPNFSSLNLRGYQNFPASRSGIRTACTLCSEILPARIKKKSVSSRNSFQVPFCSFSGAQDRFGIGISSSSKPTLVKDLGKKFTTSSLPKKPCARILFKKYFIPLKLLFSIKYPTNLVLL

SEQ ID NO: 3 - DNA, 1678 bp, Cercopithecus aethiops
cgcgagccggccgagctccagccggagctacgtgactacgtccacccgcacctacagcctaggcagcgcgctgcgccccagcaccagccgcagcctctacgcctcgtccccgggcggcgtgtatgccacgcgctcctcggccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggcggagctcgagcagctcaagggccagggcaagtcgcgcctgggggacctctacgaggaggagatgcgggagctgcgccggcaggtggaccagctaaccaacgacaaagcccgcgtcgaggtggagcgcgacaacctgaccgaggacatcatgcgcctccgggagaaattgcaggaggagatgcttcagagagaggaagccgagaacaccctgcaatctttcagacaggatgttgacaacgcgtctctggcacgtcttgaccttgaacgcaaagtggaatctttgcaagaagagattgcctttttgaagaaactccacgaagaggaaatccaggagctgcaggctcagattcaggaacagcatgtccagatcgatgtggatgtttccaagcctgacctcacggctgccctgcgtgacgtacgtcagcaatatgaaagtgtggctgccaagaacctgcaggaggcagaagaatggtacaagtccaagtttgctgacctctccgaggctgccaaccggaacaatgacgccctacgccaggcaaagcaggagtccaatgaataccggagacaggtgcagtccctcacctgtgaagtggatgcccttaaaggaactaatgagtccctggaacgccagatgcgtgaaatggaagagaactttgccgttgaagctgctaactaccaagacactattggccgtctgcaagatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggtggacacccactcaaaaaggacacttctgattaagacggttgaaactagagatggacaggttatcaacgaaacttctcagcatcacgatgaccttgaataaaaattgcatacactcagtgcagcaatatattaccagcaagaataaaaaagaaatccatatcttaaagaaacagctttcaagtgcctttctgcagtttttcaggagcgcaagatagatttggaataggaataagctctagttcttaacaaccaacactcctaaaagatttagaaaaaagtttacaacataatctagtttacagaaaaatcttgtgctagaatactttttaaaaggtattttgaatatcattaaaactgctttttttccagcaagtatctgaccaactttttctgcttcaataaatctttggaaaaattaaaaaaaaaaaaaaaaaaa

SEQ ID NO: 4 - protein, 551 aa, Cercopithecus aethiops
ASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLTEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESNEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLEKLHTLSAAIYYQQEKRNPYLKETAFKCLSAVFQERKIDLEEALVLNNQHSKIKKVYNIIFTEKSCARILFKRYFEYHNCFFSSKYLTNFFCFNKSLEKLKKKKKK

SEQ ID NO: 5 - DNA, 5193 bp, Sus scrofa
atggacttcctgctcctgctcctcctcctggcttcatctgctctagcaggcctggcctcgtggacggtttccagccccgagaccgtgcagggcatcaagggctcctgcctcatcatcccctgcaccttcggcttcccggccaacgtggaggtgccccatggcatcacagccatctggtactatgactactcaggcaagcgcctggtagtgagccactccaggaacccaaaggtggtggagaaccacttccaaggccgggccctgctgttggggcaggttgaacagaggacgtgcagcctgctgctgaaggacctgcagccccaggactcgggctcctataacttccgctttgagatcagcgagggcaaccgctggtcagatgtcaaaggcacagttgtcaccgtgacagaggtgcccagcgtgcccaccattgccttgccagccaagctgcatgagggcatggaggtggacttcaactgctccactccctatgtgtgcccgacggagccggtcaacctacagtggcaaggccaggatcccacccgctccgtcacctcccacctccagaagcttgagccctcgggcaccagccacatggagaccctgcacatggccctgtcctggcaggaccatggccggatcctgagctgccaggtctcagcagccgaacgcaggatgcagaaggagattcacctccaagtgcagtatgcccccaagggtgtggagatccttttcagccactccggacggaacgtccttccaggtgatctggtcaccctcagctgccaggtgaatagcagcaaccctcaggtcagttccgtgcagtgggtcaaggatgggacgaagctcaaagaccagaaacgtgtactgcagttgcgccgggcagcctgggctgatgctggcgtctacacctgccaagccgggaatgccgtgggctcttcagtctcacccccggtcagcctccacgtcttcatggctgaggtccaggtaagccctgtgggctccatcctggagaaccagacggtgacgctggcctgcaatacacctaaggaagcgcccagcgagctgcgctacagctggtacaagaaccacgccctgctggagggctctcacagccgcaccctccggctgcactcagttaccagggcggattcgggcttctacttctgcgaggtgcagaacgcccggggcagagagcgctctccccctgtcagcgtggtggtcagccacccacccctcaccccggacctaactgccttcctggagacacaggcggggctggtgggcatcctccaatgctctgtggtcagcgagcccccagctactctggtgttgtcacacgggggcctcatcttggcctctacctccggggagggtgaccacagcccacgcttcagtgtcgcctctgcccccaactccctgcgcctggagattcaagacctggggccaacagacagtggggaatacatgtgctcagccagcagttctcttgggaatgcgtcctccaccctggacttccatgccaatgcagcccgcctcctcatcagcccagcagcagaggtggtggaagggcaggcggtgacactgagctgcaggagcagcctgagcctgatgcctgacacccgtttttcctggtacctgaacggggccctgattctcgaggggcccagcagcagcctcctgctcccagcagcctccagcacagatgccggctcataccactgccgggcccagaacagccacagcaccagcgggccctcctcacctgctgttctcaccgtgctctacgccccacgccagcccgtgttcactgcccagctggaccctgatactgcaggagctggggccggacgccaaggcctcctcttgtgccgtgtggacagcgaccccccagcccagctgcagctgctccacaggggccgtgttgtggcctcttctctgtcatgggggggcggctgctgcacctgcggaggctgtttccaccgcatgaaggtcaccaaagcacccaacctactgcgtgtagagatccgagacccggtgctggaggatgagggtgtgtacctgtgcgaggccagcagcgccctgggcaacgcctccgcctctgcaaccttggatgcccaggccactgtcctggtcatcacaccgtcacacacgctgcaggaaggcattgaagccaacctgacttgcaacgtgagccgtgaagccagcggccctgccaacttctcctggttccgagatggggcgctatgggcccagggccctctggacaccgtgacgctgctacctgtggccagaactgatgctgccctctatgcttgccgcatcgtcaccgaggctggtgctggcctctccacccctgtggccctgaatgtgctctatccccccgatcctccaaagttgtcagccctcctggacgtggaccagggccacacggctgtgttcgtctgtactgtggacagtcgccctcttgcccagttggccctgttccgtggggaacacctcctggccgccagctcggcactccggctcccccctcgtggccgcctccaggccaaagcctcggccaactccttgcagctagaggtccgagacttgagccttggggactctggcagctaccactgtgaggccaccaacatccttggatcagccaacacttctcttaccttccaggtccgaggagcctgggtccgggtgtcaccgtcgcctgagctccaggagggccaggctgtggtcctgagctgccaggtacccataggggtcctggaggggacctcatatcgttggtatcgggatggccagcccctccaggagtccacttcggccacgctccgttttgcagccataactctgagccaggctggagcctaccattgccaagcccaagctccaggctcagccaccacggacctggctgcccctgtcagcctccacgtgacctacgcacctcgccaggccacactcaccaccctgatggactcaggcctcgggcgactgggcctccttctgtgccgtgtgaacagtgaccctcctgcccagctccgactgctccatgggagccgcctcgtggcctctactctacaaggtgtggaggagcttgcaggcagctctccccgcctacaggtggccacagcccccaacacgctgcgcctggagatccacaacgcagtgctggaggatgaaggcgtctacacctgcgaggccaccaacaccctgggtcagaccttggcctccgccgccttcgatgcccaggctatgagagtgcaggtgtggcccaatgccaccgtgcaagaggggcagctggtgaacctgacctgccttgtatggaccacgcacctggcccagctcacctacacgtggtaccgagaccagcagcagctcccaggtgctgcccactccatcctcctgcccaatgtcactgtcacagatgccgcctcctaccgctgtggcatattgatccctggccaggcactccgcctctccagacctgtcgccctggatgtcctctacgcaccccgcagactgcgcctgacccatctcttggagagccgtggtgggcagctggccgtggtgctgtgcactgtggacagtcgcccagctgcccagctgaccctcagccatgctggccgcctcctggcctcctcaaccgcagcctctgtccccaacaccctgcgcctggagctgtgggagccccggcccagtgatgagggtctctacagctgctcggcccgcagtcctctgggccaggccaacacatccctggagctgcggctagagggcgtgcaggtggcactggctccatcggccactgtgccggagggggcccctgtcacagtgacctgtgaagaccctgctgcccgcccacccactctctatgtctggtaccacaacagccgttggctgcaggaggggtcggctgcctccctctcgtttccagcggctacacgggctcacgcgggcgcctatacctgccaggtccaggatgcccagggcacacgcatctcccagcccgcagcactgcacatcctctatgcccctcgggatgctgtcctttcctccttctgggactcaagggccagccctatggccgtggtacagtgcactgtggacagcgagccacctgccgagatgaccctgtcccatgatggcaaggtgctggccaccagccatggggtccacggcttagcagtggggacaggccatgtccaggtggcccgcaacgccctgcagctgcgggtgcagaatgtgccctcacgtgacaaggacacctacgtctgcatggaccgcaactccttgggctcagtcagcaccatggggcagctgcagccagaaggtgtgcacgtggtagctgagccagggctggatgtgcctgaaggcacagcgctgaacctgagctgtcgcctccctagtggccctgggcacataggcaactccacctttgcttggttccggaacggtcggcagctacacacagagtctgtgcccacccttaccttcacccatgtggcccgcgcccaagctggcttgtaccactgccaggctgagctccccgccggggctgccacctctgctccagtcttgctccgggtgctctaccctcccaagacgcccaccatgactgtttttgtggagcccgagggtggcatccagggcattctggactgccgagtggacagtgagcccctagccagcctgaccctccacctgggcagtcggctggtggcctccagccagcctcaggctgcccctgccaagccgcacatccgcgtctcagccagtcccaatgccttgcgagtggacatggaggagctgaagcccagtgaccagggggagtatgtgtgctcggcctccaatgccctgggctctgcctctgctgccacctacttcggaaccagagccctgcatcgcctgcatctgttccagcaccttctctggttcctggggctgctggcgagcctcctcttcctactgttgggcctgggggtctggtacgcctggagacggggaaatttttacaagctgagaatgggcgaatattcagtagagatggtatctcggaaggaaaccacgcagatgtccactgaccaggaagaagttactggaatcggtgatgatgcgggctctgtgaaccaggcggcatttgatcctgcccacctctgtgaaaacacacagtctgtgaaaagcacagtctga

SEQ ID NO: 6 - protein, 1730 aa, Sus scrofa
MDFLLLLLLLASSALAGLASWTVSSPETVQGIKGSCLIIPCTFGFPANVEVPHGITAIWYYDYSGKRLVVSHSRNPKVVENHFQGRALLLGQVEQRTCSLLLKDLQPQDSGSYNFRFEISEGNRWSDVKGTVVTVTEVPSVPTIALPAKLHEGMEVDFNCSTPYVCPTEPVNLQWQGQDPTRSVTSHLQKLEPSGTSHMETLHMALSWQDHGRILSCQVSAAERRMQKEIHLQVQYAPKGVEILFSHSGRNVLPGDLVTLSCQVNSSNPQVSSVQWVKDGTKLKDQKRVLQLRRAAWADAGVYTCQAGNAVGSSVSPPVSLHVFMAEVQVSPVGSILENQTVTLACNTPKEAPSELRYSWYKNHALLEGSHSRTLRLHSVTRADSGFYFCEVQNARGRERSPPVSVVVSHPPLTPDLTAFLETQAGLVGILQCSVVSEPPATLVLSHGGLILASTSGEGDHSPRFSVASAPNSLRLEIQDLGPTDSGEYMCSASSSLGNASSTLDFHANAARLLISPAAEVVEGQAVTLSCRSSLSLMPDTRFSWYLNGALILEGPSSSLLLPAASSTDAGSYHCRAQNSHSTSGPSSPAVLTVLYAPRQPVFTAQLDPDTAGAGAGRQGLLLCRVDSDPPAQLQLLHRGRVVASSLSWGGGCCTCGGCFHRMKVTKAPNLLRVEIRDPVLEDEGVYLCEASSALGNASASATLDAQATVLVITPSHTLQEGIEANLTCNVSREASGPANFSWFRDGALWAQGPLDTVTLLPVARTDAALYACRIVTEAGAGLSTPVALNVLYPPDPPKLSALLDVDQGHTAVFVCTVDSRPLAQLALFRGEHLLAASSALRLPPRGRLQAKASANSLQLEVRDLSLGDSGSYHCEATNILGSANTSLTFQVRGAWVRVSPSPELQEGQAVVLSCQVPIGVLEGTSYRWYRDGQPLQESTSATLRFAAITLSQAGAYHCQAQAPGSATTDLAAPVSLHVTYAPRQATLTTLMDSGLGRLGLLLCRVNSDPPAQLRLLHGSRLVASTLQGVEELAGSSPRLQVATAPNTLRLEIHNAVLEDEGVYTCEATNTLGQTLASAAFDAQAMRVQVWPNATVQEGQLVNLTCLVWTTHLAQLTYTWYRDQQQLPGAAHSILLPNVTVTDAASYRCGILIPGQALRLSRPVALDVLYAPRRLRLTHLLESRGGQLAVVLCTVDSRPAAQLTLSHAGRLLASSTAASVPNTLRLELWEPRPSDEGLYSCSARSPLGQANTSLELRLEGVQVALAPSATVPEGAPVTVTCEDPAARPPTLYVWYHNSRWLQEGSAASLSFPAATRAHAGAYTCQVQDAQGTRISQPAALHILYAPRDAVLSSFWDSRASPMAVVQCTVDSEPPAEMTLSHDGKVLATSHGVHGLAVGTGHVQVARNALQLRVQNVPSRDKDTYVCMDRNSLGSVSTMGQLQPEGVHVVAEPGLDVPEGTALNLSCRLPSGPGHIGNSTFAWFRNGRQLHTESVPTLTFTHVARAQAGLYHCQAELPAGAATSAPVLLRVLYPPKTPTMTVFVEPEGGIQGILDCRVDSEPLASLTLHLGSRLVASSQPQAAPAKPHIRVSASPNALRVDMEELKPSDQGEYVCSASNALGSASAATYFGTRALHRLHLFQHLLWFLGLLASLLFLLLGLGVWYAWRRGNFYKLRMGEYSVEMVSRKETTQMSTDQEEVTGIGDDAGSVNQAAFDPAHLCENTQSVKSTV

SEQ ID NO: 7 - DNA, 1463 bp, Sus scrofa
gtcccggactccgacgagtggtagccccaggatgggtgagtttaacgagaagaagacaacatgtggcaccgtttgcctcaagtacctgctgtttacctacaactgctgcttctggctggccggcctggctgtcatggcagtgggcatctggacgctggccctcaagagtgactacatcagcctcctggcctcgggcacctacctggccacagcctacatcctggtggtggcgggcgctgtcgtcatggtgaccggggtcttgggctgctgtgccaccttcaaggagcgtcggaacctgctgcgcctgtacttcatcctgctcctcatcatctttctcctggagactcaaggtcgtgtcctcgcctatgtctactaccagcagctgaacacagagctcaaggagaaccttaaggacaccatggccaagcgctaccaccagccgggtcacgaggccgtgaccagcgctgtggaccaactgcagcaggagttccactgctgtggcagcaacaactcacaggactggcgggacagtgagtggatccgcttaagggaagcccgtggccgcgtggtccccgatagctgctgcaagacggtggtggctggttgtgggcagcgggaccacgccttcaacatttacaaggtggagggcggcttcatcaccaagtgggagaccttcatccaggagcacctcagggtcattggggctgtggggactggcattgcctgtgtgcaggtctttggcatgatcttcacatgctgcctgtacaggagcctcaagctggagcactactgaccctgccctgggcttggccgcggctctgtgctttgctgctgctgcacccaactactgagctgagaccactgagtaccaggggctgggctccctgatgacacccaccctgtgccatcaccataactttggggaccccaaccccagaggcaagcttcaagtgcctttcgctgcacaccaaagcccagcagggaagtgaggggggctggcgggacgacggtatcgggggtgttttgtggggctgcctgaacacattctgcctggtggtcagatgcaggctagccggggccttgctgagtagggcaaggccgagtgttcccagcagggggagaagcccttcacatcccaggcccttcagggattagggctttgccttgcagccacatggccccatcccagtttgagaagctgagtaagctctgacccttgggcctgggcctctgcccctccccacccaggcctcgtctccctcagagcccctgctgtcttccccaccgcagtcaccaccacccgaaatgccacatggtcacttgtgcactgccccgtccatgtgcctgtgtggggcaggggcctcccggttttgttcactgctgtacccagatgcctacaaccatccctgccacatacaggtgctcaataaacacttgtggggcagatggacgaaaaaaaaaaa

SEQ ID NO: 8 - protein, 254 aa, Sus scrofa
MGEFNEKKTTCGTVCLKYLLFTYNCCFWLAGLAVMAVGIWTLALKSDYISLLASGTYLATAYILVVAGAVVMVTGVLGCCATFKERRNLLRLYFILLLIIFLLEIIAGVLAYVYYQQLNSELKENLKDTMAKRYHCFGHEAVTSAVDQLCQEFHCCGSNNSQDWRDSEWIRLREARGRVVPDSCCKTVVAGCGQRDHAFHNIYKVEGGFITKLETFIQEHLRVIGAVGTGIACVQVFGMIFTCCLYRSLKLEHY

(19) United States
(12) Patent Application Publication
(10) Pub. No.: US 2006/0110762 A1
(43) Pub. Date: May 25, 2006

(54) Porcine reproductive and respiratory syndrome virus receptor components and uses thereof

(76) Inventors: Sanjay Kapil, Manhattan, KS (US); Jeong-Ki Kim, Manhattan, KS (US)

Correspondence Address: FISH & RICHARDSON P.C., PO BOX 1022, MINNEAPOLIS, MN, 55440-1022, US

(21) Appl. No.: 11/270,040

(22) Filed: Nov. 9, 2005

Related U.S. Application Data
(60) Provisional application No. 60/680,297, filed on May 12, 2005.
(60) Provisional application No. 60/626,788, filed on Nov. 10, 2004.

(51) Int. Cl. C12Q 1/70; C12Q 1/68

(52) U.S. Cl. 435/6; 435/5

(57) ABSTRACT

This invention relates to host cellular receptor components for Porcine Reproductive and Respiratory Syndrome Virus (PRRSV) and methods of their use in diagnosis, prevention, control, and treatment of PRRSV disease in animals.

### CROSS REFERENCE TO RELATED APPLICATIONS

This application claims priority to U.S. Provisional Patent Application Ser. No. 60/680,297, filed May 12, 2005, and U.S. Provisional Patent Application Ser. No. 60/626,788, filed Nov. 10, 2004, the contents of which are hereby incorporated by reference.

### TECHNICAL FIELD

This invention relates to host cellular receptor components for Porcine Reproductive and Respiratory Syndrome Virus (PRRSV) and methods of their use in diagnosis, prevention, control, and treatment of PRRSV in swine. More particularly the invention relates to vimentin and its use for diagnosis, prevention, control, and treatment of PRRSV in swine.

### BACKGROUND

Porcine reproductive and respiratory syndrome (PRRS) was first reported in the U.S.A. in 1987. The causative agent of PRRS, porcine reproductive and respiratory syndrome virus (PRRSV), has had an enormous impact on the global swine industry (Holck and Polson, 2003, In: 2003 PRRS Compendium, eds., Zimmerman and Yoon, Pgs., 51-58). The disease is characterized by reproductive failure in sows and gilts, pneumonia in young growing pigs, and an increase in preweaning mortality. PRRSV was identified first in Europe and then in the U.S.A. The European strain of PRRSV, designated as Lelystad virus (LV), has been cloned and sequenced (see, e.g., U.S. Pat. No. 6,773,908). PRRSV is classified in the Arteriviridae family.

The PRRSV genome is a positive sense RNA molecule of about 15 kb. The viral genome is flanked by a 5′ end-untranslated region (UTR) of 156-220 nucleotides, and a 3′ end-UTR of 59-117 nucleotides. The viral genome consists of eight overlapping open reading frames encoding non-structural, replication, and structural proteins. ORFs 1a and 1b likely encode viral RNA polymerase. ORFs 5, 6 and 7 encode a glycosylated membrane protein (E), an unglycosylated membrane protein (M) and a nucleocapsid protein (N), respectively. ORFs 2 to 4 appear to have the characteristics of membrane-associated proteins. However, the translation products of ORFs 2 to 4 were not detected in virus-infected cell lysates or in virions (U.S. Pat. No. 6,773,908).

The genomic organization of arteriviruses resembles coronaviruses and toroviruses in that their replication involves the formation of a 3′-coterminal nested set of subgenomic mRNAs (sg mRNAs). Arteriviruses replicate in the cytoplasm of their host cells, usually macrophages. Genomic RNA is synthesized via full-length, negative-sense replicative intermediate. PRRSV virions bud through membranes of the endoplasmic reticulum into intracellular vesicles; from there they move to the surface of the cell in vesicles and are released by exocytosis. The primary target cells of the PRRSV are porcine alveolar macrophages. PRRSV replicates in the macrophages of infected pigs, entering the cells by receptor-mediated endocytosis.

The disease caused by PRRSV has been identified by the U.S. National Pork Board as the most serious infectious disease currently facing pork producers. PRRSV causes severe reproductive failure in sows and pneumonia in growing pigs, resulting in slow and stunted growth. The reproductive failure is characterized by abortions, stillbirths, and the birth of weak piglets that often die soon after birth of respiratory disease and secondary infections. Older pigs may demonstrate mild signs of respiratory disease, sometimes complicated by secondary infections. Annual farm losses from PRRS are estimated at $600 million in the U.S.A. alone. The virus spreads quickly in native swine populations, with up to 95% of swine in a herd becoming seropositive within 2-3 months after an introduction.

PRRSV is known to grow in cell lines such as CL-2621, MA-104, MARC-145, and in primary cultures of porcine alveolar macrophages. CL-2621, MA-104, and MARC-145 are continuous cell lines of simian origin. See, U.S. Pat. No. 5,476,778. Development of better vaccines has been hampered by the lack of susceptible cell lines that allow PRRSV to be propagated to high titers. Another obstacle is posed by the genetic variability of PRRSV, as there are significant antigenic and genetic variations among the PRRSV isolates in the United States and Europe (Ropp et al., 2004, J. Virol., 78: 3684-3703). Further, there are limitations or risks of using vaccines derived from simian cells.

### SUMMARY

The present invention is based on the discovery of host cellular receptor components for PRRSV. This discovery permits the development of methods for diagnosing, preventing, and treating PRRS in swine.

In one aspect, the invention features a non-simian, mammalian cell whose genome includes a recombinant nucleic acid construct. The construct includes a nucleic acid having about 90% or greater sequence identity to the sequence set forth in SEQ ID NO:1 or SEQ ID NO:3. The nucleic acid can have the sequence set forth in SEQ ID NO: 1, or the sequence set forth in SEQ ID NO:3. The nucleic acid construct can further include an expression control sequence operably linked to the nucleic acid. The nucleic acid further can include a nucleic acid having about 90% or greater sequence identity to the sequence of a porcine or simian CD 151 coding sequence, such as the nucleotide sequence set forth in SEQ ID NO:7, operably linked to an expression control sequence. The cell can be a swine testicular cell line or a primary swine kidney cell line.

The invention also features a method for making a virus preparation. The method includes infecting a culture of a non-simian cell as described herein with PRRS virus (e.g., the Lelystad strain or the VR 2332 strain of PRRSV), incubating the culture until at least 50% of the cells exhibit one or more cytopathic effects, lysing the cells, and harvesting PRRS virus from the lysed cells to produce the virus preparation. The method can further include the step of purifying PRRS virus, after the harvesting step, to produce the virus preparation. The method can also include the step of inactivating the virus, after the harvesting step, to produce a killed virus preparation. A vaccine can be made from PRRS virus prepared by such methods. In another aspect, the virus preparation can be administered to a pig, either male or female, in order to treat a PRRSV infection. The virus preparation can be administered to a tissue or organ including the nasal passages, the throat, the vagina, and the lungs.

In another aspect, the invention features a method for treating a PRRSV infection. The method includes administering a composition that includes an anti-vimentin antibody to a male or a female pig. The composition can be an ointment, a nebulizable liquid, a powder, or a sprayable liquid. The anti-vimentin antibody can be a monoclonal, e.g., 7G10, or a polyclonal antibody. The composition can be administered to a tissue or organ including the nasal passages, the throat, the vagina, and the lungs.

In another aspect, the invention features a method of screening for nucleic acid polymorphisms that are correlated with PRRSV susceptibility or resistance. The method includes determining part or all of the vimentin nucleotide sequence in a plurality of pigs, and determining whether any polymorphisms present in the vimentin nucleotide sequence are correlated with PRRSV susceptibility or resistance among the pigs. The pigs can be of two or more different inbred lines of swine, e.g., two, three, four, five, six, seven, or eight, each breed having a different susceptibility to PRRSV relative to at least one of the other of breeds.

In another aspect, the invention features an isolated nucleic acid that includes a sequence having about 90% or more sequence identity to SEQ ID NO:1, e.g., about 95% or more sequence identity to SEQ ID NO:1, or 100% sequence identity to SEQ ID NO:1. An isolated nucleic acid can be a nucleic acid encoding a polypeptide having about 90% or more sequence identity to SEQ ID NO:2, e.g., about 95% or more sequence identity to SEQ ID NO:2, or 100% sequence identity to SEQ ID NO:2.

In another aspect, the invention features an isolated nucleic acid that includes a sequence having about 90% or more sequence identity to SEQ ID NO:3. The invention also features an isolated nucleic acid that encodes a polypeptide having about 90% or more sequence identity to SEQ ID NO:4. The nucleic acid can further include a control element operably linked to the nucleic acid, e.g., a CMV promoter or an SV40 promoter. The invention also features an isolated polypeptide having about 90% or more sequence identity to SEQ ID NO:2 or SEQ ID NO:4.

In another aspect, the invention features a transgenic non-human mammal whose genome contains a nucleic acid construct that includes a control element operably linked to a nucleic acid encoding a polypeptide having about 90% or more sequence identity to SEQ ID NO:2 or 4. The nucleic acid construct further can include a control element operably linked to a nucleic acid encoding a polypeptide having 90% or greater sequence identity to the sequence set forth in SEQ ID NO:8. The non-human mammal can be a mouse. Cells can be isolated from the transgenic non-human mammal. The mammal can be produced by introducing a nucleic acid construct that includes a control element operably linked to a nucleic acid encoding a polypeptide having about 90% or more sequence identity to SEQ ID NO:2 or 4 into a non-human mammal.

In yet another aspect, the invention features a method for rendering cells susceptible to PRRSV. The method includes providing cells resistant to PRRSV and introducing a nucleic acid construct into the cells, wherein the nucleic acid construct includes a nucleic acid encoding a vimentin polypeptide. The nucleic acid can have at least about 90% sequence identity to the sequence set forth in SEQ ID NO:1 or SEQ ID NO:3.

Other features and advantages of the invention will be apparent from the following detailed description, and from the claims. Unless otherwise defined, all technical and scientific terms used herein have the meaning commonly understood by one of ordinary skill in the art to which this invention belongs. All publications, patent applications, patents, and other references mentioned herein are incorporated by reference in their entirety. In case of conflict, the present specification, including definitions, will control. The disclosed materials, methods, and examples are illustrative only and not intended to be limiting. Skilled artisans will appreciate that methods and materials similar or equivalent to those described herein can be used to practice the invention.

### DESCRIPTION OF DRAWINGS

FIG. 1 is a porcine vimentin cDNA sequence from a swine testicle (ST) cell line (SEQ ID NO:1).

FIG. 2 is the amino acid sequence of the polypeptide encoded by the cDNA of FIG. 1 (SEQ ID NO:2).

FIG. 3 is a monkey vimentin coding sequence from MARC-145 cells (SEQ ID NO:3).

FIG. 4 is the amino acid sequence of the polypeptide encoded by the coding sequence of FIG. 3 (SEQ ID NO:4).

FIG. 5 is an alignment of the porcine and monkey nucleotide sequences shown in FIG. 1 and FIG. 3.

FIG. 6 shows the nucleotide and amino acid sequences of a porcine sialoadhesion (SEQ ID NOS:5 and 6), a porcine CD151 nucleic acid (SEQ ID NO:7), and a porcine CD151 polypeptide (SEQ ID NO:8).

Like reference symbols in the various drawings indicate like elements.

### DETAILED DESCRIPTION

The present inventors have discovered that vimentin, a type III intermediate filament protein, can serve as a receptor for PRRSV entry into porcine cells. As described herein, vimentin binds to PRRSV nucleocapsid protein N, and polyclonal anti-vimentin antibodies have PRRSV neutralizing activity. Furthermore, introducing vimentin into a host cell that is non-susceptible to PRRSV renders the cell susceptible to infection. Based on these results, strategies for diagnosis, treatment and control of PRRS in pigs can be developed.

Isolated Vimentin Nucleic Acids

As used herein, an “isolated nucleic acid” refers to a nucleic acid that is separated from other nucleic acid molecules that are present in a genome, including nucleic acids that normally flank one or both sides of the nucleic acid in a genome (e.g., nucleic acids that encode non-vimentin proteins). Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences, as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. The term “isolated” as used herein with respect to nucleic acids also includes any non-naturally-occurring nucleic acid sequence since such non-naturally-occurring sequences are not found in nature and do not have immediately contiguous sequences in a naturally-occurring genome. A nucleic acid existing among hundreds to millions of other nucleic acids within, for example, cDNA libraries or genomic libraries, or gel slices containing a genomic DNA restriction digest, is not to be considered an isolated nucleic acid.

Suitable nucleic acids typically are at least about 300 nucleotides in length. For example, the nucleic acid can be about 300, 350, 500, 600, 653, 700, 500-600, 601-850 or 820-999 nucleotides (e.g., 150, 200, 250, 300, 350, 400, 450, 500, 750, 800 or 980 nucleotides, or greater than 1000 nucleotides (e.g. 1000, 1100, 1200, 1228, 1500, 2000, 2220, 2500, or 3000 nucleotides). Nucleic acids of the invention can be in a sense or antisense orientation, can be complementary to a vimentin reference sequence, and can be DNA, RNA, or nucleic acid analogs. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller, 1997, Antisense Nucleic Acid Drug Dev., 7: 187-195; Hyrup et al., 1996, Bioorgan. Med. Chem., 4: 5-23. In addition, the deoxyphosphate backbone can be replaced with, for example, a phosphorothioate or phosphorodithioate backbone, a phosphoroamidite, or an alkyl phosphotriester backbone. To improve stability of expression, codon usage may be optimized in some cases.

In one embodiment, an isolated nucleic acid includes part or all of a vimentin coding sequence such as the porcine or simian vimentin sequences set forth in SEQ ID NO:1 and SEQ ID NO:3, respectively. In another embodiment, an isolated nucleic acid includes part or all of a vimentin coding sequence that has one or more nucleotide sequence variants relative to SEQ ID NO:1 or SEQ ID NO:3. As used herein, “nucleotide sequence variant” refers to an alteration in a given sequence, and includes variations that occur in coding and non-coding regions, including exons, introns, and untranslated sequences. Nucleotides are referred to herein by the standard one-letter designation (A, C, G, or T). Variations include substitutions of one or more nucleotides, deletions of one or more nucleotides, and insertions of one or more nucleotides. As used herein, “untranslated sequence” includes 5′ and 3′ flanking regions that are outside of the mRNA as well as 5′ and 3′ untranslated regions (5′-UTR or 3′-UTR) that are part of the mRNA, but are not translated.

Certain vimentin nucleotide sequence variants do not alter the amino acid sequence. Such variants, however, could alter regulation of transcription as well as mRNA stability. Vimentin nucleotide sequence variants can occur within intron sequences. Vimentin nucleotide sequence variants that do not change the amino acid sequence also can be within an exon or within the 5′ or 3′untranslated vimentin genomic sequences.

In one embodiment, an isolated nucleic acid can have at least 90% sequence identity (e.g., at least 91%, 92%, 93%, 94%, 95%, 96%, 97%, 98%, 98.5%, 99.0%, 99.5%, 99.6%, 99.7%, 99.8%, 99.9%, or 100%) to the sequence set forth in SEQ ID NO:1 or SEQ ID NO:3. In other embodiments, nucleic acid molecules of the invention can have at least 98% (e.g., 98.5%, 99.0%, 99.5%, 99.6%, 99.7%, 99.8%, 99.9%, or 100%) sequence identity with a region of SEQ ID NO:1 or SEQ ID NO:3. A region of identity to SEQ ID NO:1 or to SEQ ID NO:3 is at least 15 nucleotides in length (e.g., 50, 60, 70, 75, 100, 150, 200, 250, 500, 750, 900, 1000, or 1200 nucleotides in length). For example, a nucleic acid molecule can have at least 75 nucleotides in common with a region of SEQ ID NO:1 or SEQ ID NO:3.

Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a query nucleic acid or amino acid sequence is compared to a subject nucleic acid or amino acid sequence using the BLAST 2 Sequences (B12seq) program from the stand-alone version of BLASTZ containing BLASTN version 2.0.14 and BLASTP version 2.0.14. This stand-alone version of BLASTZ can be obtained from the Fish & Richardson's web site (www.fr.com/blast) or the U.S. government's National Center for Biotechnology Information web site (www.ncbi.nlm.nih.gov/blast/executables). Instructions explaining how to use the B12seq program can be found in the readme file accompanying BLASTZ.

B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows:—i is set to a file containing the first nucleic acid sequence to be compared (e.g., C:\seq1.txt):—j is set to a file containing the second nucleic acid sequence to be compared (e.g., C:\seq2.txt);—p is set to blastn;—o is set to any desired file name (e.g., C:\output.txt);—q is set to −1;—r is set to 2; and all other options are left at their default setting. The following command will generate an output file containing a comparison between two sequences: C:\B12seq—i c:\seq1.txt—j c:\seq2.txt—p blastn—o c:\output.txt—q −1—r 2. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides from the target sequence presented in alignment with sequence from the identified sequence starting with any matched position and ending with any other matched position. A matched position is any position where an identical nucleotide is presented in both the target and identified sequence. Gaps presented in the target sequence are not counted since gaps are not nucleotides. Likewise, gaps presented in the identified sequence are not counted since target sequence nucleotides are counted, not nucleotides from the identified sequence.

The percent identity over a particular length is determined by counting the number of matched positions over that length and dividing that number by the length, followed by multiplying the resulting value by 100. For example, if (1) a 1000 nucleotide target sequence is compared to the sequence set forth in SEQ ID NO:1, (2) the B12seq program presents 200 nucleotides from the target sequence aligned with a region of the sequence set forth in SEQ ID NO:1 where the first and last nucleotides of that 200 nucleotide region are matches, and (3) the number of matches over those 200 aligned nucleotides is 180, then the 1000 nucleotide target sequence contains a length of 200 and a percent identity over that length of 90 (i.e., 180÷200×100=90).

It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

Isolated nucleic acid molecules can be produced by standard techniques. For example, polymerase chain reaction (PCR) techniques can be used to obtain an isolated nucleic acid containing a vimentin nucleotide sequence. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length (e.g., 10, 15, 20, 25, 27, 34, 40, 45, 50, 52, 60, 65, 70, 75, 82, 90, 102, 150, 200, 250 nucleotides in length). General PCR techniques are described, for example in PCR Primer: A Laboratory Manual, Ed. by Dieffenbach, C. and Dveksler, G., Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication or nucleic acid sequence-based amplification also can be used to obtain isolated nucleic acids. See, for example, Lewis, 1992, Genetic Engineering News, 12: 1; Guatelli et al., 1990, Proc. Natl. Acad. Sci. USA, 87: 1874-1878; and Weiss, 1991, Science, 254: 1292.

Isolated nucleic acids of the invention also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

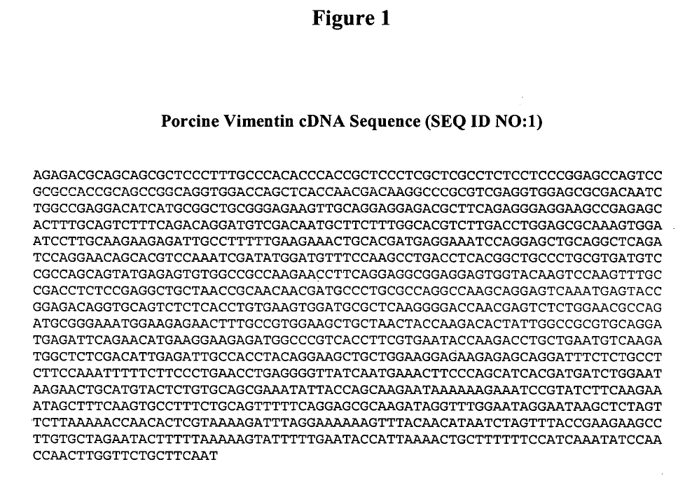

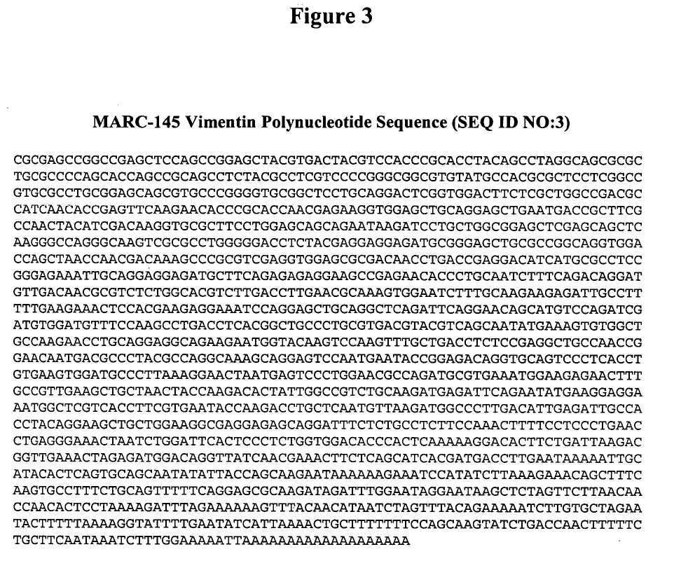

Isolated nucleic acids of the invention also can be obtained by mutagenesis. For example, the sequence depicted in FIG. 1 or 3 can be mutated using standard techniques including oligonucleotide-directed mutagenesis and site-directed mutagenesis through PCR. See, Short Protocols in Molecular Biology, Chapter 8, Green Publishing Associates and John Wiley & Sons, Edited by Ausubel, F. M et al., 1992.

Recombinant Nucleic Acid Constructs

The invention also provides recombinant nucleic acid constructs containing vimentin nucleic acids described above and nucleic acid constructs that include non-vimentin nucleic acids (e.g., a porcine CD151 coding sequence set forth in SEQ ID NO:7, a simian CD151 nucleic acid, a nucleic acid having at least 90% identity to the nucleic acid sequence of SEQ ID NO:7 or 90% identity to a simian CD151 nucleic acid sequence, or a porcine sialoadhesion nucleic acid (e.g., SEQ ID NO:5). In some embodiments, a nucleic acid construct includes both a vimentin nucleic acid and a porcine or simian CD151 nucleic acid. As used herein, a “nucleic acid construct” is a replicon, such as a vector, plasmid, phage, or cosmid, into which another DNA segment may be inserted so as to bring about the replication of the inserted segment. The nucleic acid constructs of the invention can be expression vectors. An “expression vector” is a nucleic acid construct that includes one or more expression control sequences, and an “expression control sequence” is a DNA sequence that controls and regulates the transcription and/or translation of another DNA sequence.

In the nucleic acid constructs of the invention, the nucleic acid of interest can be operably linked to one or more expression control sequences. As used herein, “operably linked” means incorporated into a nucleic acid construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters enhancers can function when located at various distances from the transcription site. An enhancer also can be located downstream from the transcription initiation site. In addition, an enhancer modulates transcription in an orientation-independent manner. This feature of enhancers differs from other regulatory DNA sequences, which must to be positioned in a certain orientation in relation to a gene of interest in order to exert their transcription promoting or repressing effect. A coding sequence is “operably linked” and “under the control” of expression control sequences in a cell when RNA polymerase is able to transcribe the coding sequence into mRNA, which then can be translated into the protein encoded by the coding sequence. Suitable expression control sequences include a cytomegalovirus promoter (CMV) promoter or an SV40 virus promoter.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

A nucleic acid construct can include a tag sequence designed to facilitate subsequent manipulation of the expressed nucleic acid sequence (e.g., purification or localization). Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide including at either the carboxyl or amino terminus. A nucleic acid construct also can include sequences encoding a selectable marker (e.g., an antibiotic resistance gene conferring resistance to kanamycin, G418, bleomycin, or hygromycin) for use in selecting cell lines.

The invention also features host cells containing nucleic acid constructs described herein. The term “host cell” is intended to include prokaryotic and eukaryotic cells into which a recombinant expression vector can be introduced. A host cell can be a primary culture or a continuous culture. Eukaryotic cells can be, for example, mammalian cells such as human, simian, porcine, feline, canine, bovine, or rodent cells. Non-simian, mammalian host cells are particularly useful for diagnosis, treatment, and control of PRRS in pigs. For example, human, rodent, bovine, or porcine cell lines such as swine testicular cells or porcine embryonic kidney cells are particularly useful non-simian, mammalian host cells. An example of a suitable porcine cell line is the kidney cell line designated PK15. Another suitable cell line is BHK21. It is contemplated that the presence of one or both of vimentin and CD151 nucleic acids in a non-simian, mammalian cell line can convert a PRRSV-resistant cell line into a PRRSV-susceptible cell line. For example, swine testicular (ST) cells are normally not susceptible to PRRSV transfection in vitro. However, a ST cell line can be converted to a susceptible line by introducing a recombinant nucleic acid that permits expression of a simian vimentin polypeptide in such cells and, in some embodiments, a recombinant nucleic acid that permits expression of a porcine or simian CD151 coding sequence in such cells. As described in Example 10, a non-susceptible cell line (BHK-21) was rendered susceptible to PRSSV by introducing recombinantly produced simian vimentin into the cells. Recombinant protein was introduced using the Chariot™ protein delivery system (Active Motif, Inc., Carlsbad, Calif.).

As used herein, “transformed” and “transfected” encompass the introduction of a nucleic acid construct (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Methods for transforming and transfecting host cells are found in Sambrook et al., Molecular Cloning: A Laboratory Manual (2^{nd} edition), Cold Spring Harbor Laboratory, New York (1989), and U.S. Pat. Nos. 5,580,859 and 5,589,466.

Vimentin Polypeptides

Isolated vimentin polypeptides are provided herein (see, for example, SEQ ID NO:2 and SEQ ID NO:4). The term “polypeptide” refers to a chain of at least four amino acid residues (e.g., 4-8,9-12, 13-15, 16-18, 19-21, 22-100, 100-150, 150-200, 200-300, or a full-length vimentin polypeptide). Vimentin polypeptides may or may not bind PRRSV, or may have a binding capacity or activity that is altered relative to the reference vimentin polypeptide. Polypeptides that do not bind, or that have an altered binding capacity or activity are useful for diagnostic purposes (e.g., for producing antibodies having specific binding affinity for variant vimentin polypeptides). In some embodiments, a vimentin polypeptide has post-transcriptional modifications, e.g., is phosphorylated.

The term “isolated” with respect to a vimentin polypeptide refers to a polypeptide that has been separated from cellular components that naturally accompany it. Typically, the polypeptide is isolated when it is at least 60% (e.g., 70%, 80%, 90%, 95%, or 99%), by weight, free from proteins and naturally occurring organic molecules that are naturally associated with it. In general, an isolated polypeptide will yield a single major band on a non-reducing polyacrylamide gel.

Isolated polypeptides can be obtained, for example, by extraction from a natural source (e.g., testicular tissue), chemical synthesis, or by recombinant production in a host cell. Vimentin polypeptides obtained from commercial sources typically contain multiple bands after electrophoresis on denaturing polyacrylamide gels. Thus, further purification is necessary to obtain an isolated vimentin from commercial preparations. To recombinantly produce vimentin polypeptides, a nucleic acid containing a vimentin nucleotide sequence can be ligated into an expression vector and used to transform a bacterial or eukaryotic host cell (e.g., insect, yeast, or mammalian cells). Suitable vectors for mammalian cells include the pcDNA™ vectors sold by Invitrogen™. In bacterial systems, a strain of Escherichia coli such as BL-21 or M15 can be used. Suitable E. coli vectors include the pGEX series of vectors that produce fusion proteins with glutathione S-transferase (GST) and the pQE-30 series of vectors that produce fusion proteins with 6×His tags. Depending on the vector used, transformed E. coli are typically grown exponentially, then stimulated with isopropylthiogalactopyranoside (IPTG) prior to harvesting. In general, expressed fusion proteins are soluble and can be purified easily from lysed cells by adsorption to glutathione-agarose beads followed by elution in the presence of free glutathione. The pGEX vectors are designed to include thrombin or factor Xa protease cleavage sites so that the cloned target gene product can be released from the GST moiety. Alternatively, 6×His-tags can be used to facilitate isolation via immobilized metal affinity chromatography on a nickel-nitrilotriacetic acid (Ni-NTA) column.

Isolated vimentin polypeptides include amino acid sequence variants relative to another vimentin polypeptide.

In eukaryotic host cells, a number of viral-based expression systems (described herein) can be utilized to express vimentin polypeptides. A nucleic acid encoding a polypeptide of the invention can be cloned into, for example, a baculoviral vector such as pBlueBac (Invitrogen, Carlsbad, Calif.) and then used to co-transfect insect cells such as Spodoptera frugiperda (Sf9) cells with wild type DNA from Autographa californica multiply enveloped nuclear polyhedrosis virus (AcMNPV). Recombinant viruses producing polypeptides of the invention can be identified by standard methodology.

Mammalian cell lines that stably express vimentin polypeptides can be produced by using expression vectors with the appropriate control elements and a selectable marker. For example, the pQE-30 expression vector (Qiagen, Valencia, Calif.) is suitable for expression of vimentin polypeptides in cells such as, Chinese hamster ovary (CHO) cells, COS-1 cells, human embryonic kidney 293 cells, NIH3T3 cells, BHK21 cells, MDCK cells, ST cells, PK15 cells, or human vascular endothelial cells (HUVEC). In some instances, the pQE-30 vector can be used to express porcine vimentin in BHK21 cells, where the vector includes a CMV promoter and a G418 antibiotic resistance gene. In other instances, the pQE-30 vector can be used to express a simian vimentin (such as, for example, the one set forth in SEQ ID NO:3) in MARC-145 cells, where the vector includes a CMV promoter and a G418 antibiotic resistance gene.

Following introduction of the expression vector, stable cell lines can be selected, e.g., by antibiotic resistance to G418, kanamycin, or hygromycin. Alternatively, amplified sequences can be ligated into a mammalian expression vector such as pQE-30 (Qiagen, Valencia, Calif.) and then transcribed and translated in vitro using wheat germ extract or rabbit reticulocyte lysate.

Vimentin Sequence Polymorphisms

It is known that certain genes or regions of a genome have sequence polymorphisms. Sequence polymorphisms, e.g., single nucleotide polymorphisms (SNPs), simple sequence repeats (SSRs) and restriction fragment length polymorphisms (RFLPs) can aid in the genotyping of plants and animals, and be used as markers for marker-assisted breeding. Such polymorphisms may exist in and around the vimentin locus in pigs. Thus, the invention features a method of screening for nucleic acid polymorphisms that are correlated with PRRSV susceptibility or resistance. The method includes determining part or all of the vimentin nucleotide sequence in a plurality of pigs. Vimentin nucleotide sequence and its variants can be detected, for example, by sequencing exons, introns, 5′ untranslated sequences, or 3′ untranslated sequences, by performing allele-specific hybridization, allele-specific restriction digests, mutation specific polymerase chain reactions (MSPCR), by single-stranded conformational polymorphism (SSCP) detection, denaturing high performance liquid chromatography, infrared matrix-assisted laser desorption/ionization (IR-MALDI) mass spectrometry (WO 99/57318), denaturing gradient gel electrophoresis, and combinations of such methods.

Genomic DNA generally is used in the analysis of vimentin nucleotide sequences. Genomic DNA is typically extracted from a biological sample such as a liver sample, but can be extracted from other biological samples, including tissues, such as, for example, ear notch biopsies or hair clippings. Routine methods can be used to extract genomic DNA from a blood or tissue sample, including, for example, phenol extraction. Alternatively, genomic DNA can be extracted with kits such as the QIAamp® Tissue Kit (Qiagen, Chatsworth, Calif.), Wizard® Genomic DNA purification kit (Promega, Madison, Wis.) and the A.S.A.P.™ Genomic DNA isolation kit (Boehringer Mannheim, Indianapolis, Ind.).

Typically, an amplification step is performed before proceeding with the detection method. For example, exons or introns of the vimentin gene can be amplified, and then directly sequenced. Dye primer sequencing can be used to increase the accuracy of detecting heterozygous samples.

Allele specific hybridization also can be used to detect vimentin nucleotide sequence variants, including complete haplotypes of a non-human mammal. See, Stoneking et al., 1991, Am. J. Hum. Genet., 48: 370-382, and Prince et al., 2001, Genome Res., 11: 152-162. In practice, samples of DNA or RNA from one or more non-human mammals can be amplified using pairs of primers and the resulting amplification products can be immobilized on a substrate (e.g., in discrete regions). Hybridization conditions are selected such that a nucleic acid probe can specifically bind to the sequence of interest, e.g., the vimentin nucleic acid molecule containing a particular vimentin nucleotide sequence variant. Such hybridizations typically are performed under high stringency as some nucleotide sequence variants include only a single nucleotide difference. High stringency conditions can include the use of low ionic strength solutions and high temperatures for washing. For example, in some cases, nucleic acid molecules can be hybridized at 42° C. in 2×SSC (0.3M NaCl/0.03M sodium citrate/0.1% sodium dodecyl sulfate (SDS) and washed in 0.1×SSC (0.015M NaCl/0.0015M sodium citrate), 0.1% SDS at 65° C. In other cases, nucleic acid molecules can be hybridized at 42° C. in 2×SSC (0.3M NaCl/0.03M sodium citrate/0.1% sodium dodecyl sulfate (SDS) and washed in 0.1×SSC (0.015M NaCl/0.0015M sodium citrate), 0.1% SDS at 70° C. Hybridization conditions can be adjusted to account for unique features of the nucleic acid molecule, including length and sequence composition. Probes can be labeled (e.g., fluorescently) to facilitate detection. In some embodiments, one of the primers used in the amplification reaction is biotinylated (e.g., 5′ end of reverse primer) and the resulting biotinylated amplification product is immobilized on an avidin or streptavidin coated substrate.

Allele-specific restriction digests can be performed in the following manner. For vimentin nucleotide sequence variants that introduce a restriction site, a restriction digest with the particular restriction enzyme can differentiate the alleles. For vimentin nucleotide sequences that do not alter a common restriction site, mutagenic primers can be designed that introduce a restriction site when the variant allele is present or when the wild type allele is present. A portion of a vimentin nucleic acid can be amplified using the mutagenic primer and a wild type primer, followed by a digest with the appropriate restriction endonuclease.

Certain sequence variants, such as insertions or deletions of one or more nucleotides, change the size of the DNA fragment encompassing the variant. The insertion or deletion of nucleotides can be assessed by amplifying the region encompassing the variant and determining the size of the amplified products in comparison with size standards. For example, a region of vimentin nucleic acid can be amplified using a primer set from either side of the variant. One of the primers is typically labeled, for example, with a fluorescent moiety, to facilitate sizing. The amplified products can be electrophoresed through acrylamide gels with a set of size standards that are labeled with a fluorescent moiety that differs from the primer.

PCR conditions and primers can be developed that amplify a product only when the variant allele is present, or only when the wild type allele is present (MSPCR or allele-specific PCR). For example, in cases where an SNP is known to correlate with the presence of a non-susceptible allele, DNA from susceptible and non-susceptible animals can be amplified separately, using either a wild type primer or a primer specific for a non-susceptible allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous pigs, reaction products would be detected in each reaction. Porcine DNA samples containing solely one allele would have amplification products only in a reaction containing one the appropriate primer. Therefore, for example, porcine DNA samples containing solely a susceptible allele would have amplification products only in a reaction that uses a susceptible-specific primer. Allele-specific PCR also can be performed using allele-specific primers that introduce priming sites for two universal energy-transfer-labeled primers (e.g., one primer labeled with a green dye such as fluoroscein and one primer labeled with a red dye such as sulforhodamine). Amplification products can be analyzed for green and red fluorescence in a plate reader. See, Myakishev et al., 2001, Genome 11: 163-169.

Mismatch cleavage methods also can be used to detect differing sequences by PCR amplification, followed by hybridization with the wild type sequence and cleavage at points of mismatch. Chemical reagents, such as carbodiimide or hydroxylamine and osmium tetroxide can be used to modify mismatched nucleotides to facilitate cleavage.

The presence of a certain vimentin variant, corresponding, for example, to PRRSV susceptibility, can be determined based on the presence or absence of one or more sequence variants, or a variant profile. “Variant profile” refers to the presence or absence of a plurality (i.e., two or more sequence variants) of vimentin nucleotide sequence variants or vimentin amino acid sequence variants. For example, a variant profile can include the complete vimentin haplotype of the mammal or can include the presence or absence of a set of SNPs (e.g., one or more common non-synonymous SNPs that alter the amino acid sequence of a vimentin polypeptide). In some cases, a variant profile includes detecting the presence or absence of two or more non-synonymous SNPs (e.g., 2 or 3 non-synonymous SNPs). In certain cases, there may be a population-specific pharmacogenetic variation. This could occur if certain nucleotide and amino acid sequence variants were detected solely in a particular swine population.

Samples for sequence analysis are taken from two or more individual pigs, e.g., 2, 3, 4, 10, 20, 50, 100, or 150 different pigs. The pigs may be related, e.g., as parent, progeny, or sibling, or may be unrelated. The pigs may be of the same breed, or may represent two or more different pig breeds.

Once any polymorphisms present in the vimentin nucleotide sequence or genomic regions flanking the vimentin sequence are identified, a statistical analysis is carried out to determine whether a particular polymorphism is correlated with PRRSV susceptibility or with PRRSV resistance. A correlation between a particular polymorphism and PRRSV susceptibility is considered statistically significant at p≦0.05 with an appropriate parametric or non-parametric statistic, e.g., LOD test, Chi-square test, Student's t-test, Mann-Whitney test, or F-test. In some embodiments, a correlation is statistically significant at p≦0.01, p<0.005, or p<0.001.

Vimentin Polypeptide Isoforms and Detection Thereof

A vimentin polypeptide and its variants can be detected by antibodies that have specific binding affinity for a particular polypeptide or its isoforms. Vimentin polypeptides can be produced in various ways, including recombinantly, as discussed herein. Host animals such as pigs, rabbits, chickens, mice, guinea pigs and rats can be immunized by injection of a particular vimentin polypeptide. Because vimentin amino acid sequences differ among different species, it is possible to make polyclonal antibodies against variable regions of the polypeptide. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin and dinitrophenol. Polyclonal antibodies are heterogeneous populations of antibody molecules that are contained in the sera of immunized animals. Monoclonal antibodies, which are homogeneous populations of antibodies to a particular antigenic epitope, can be prepared using a vimentin polypeptide and standard hybridoma technology. In particular, monoclonal antibodies can be obtained by any technique that provides for the production of antibody molecules by continuous cell lines in culture such as described by Kohler et al., 1975, Nature, 256: 495, the human B-cell hybridoma technique (Kosbor et al., 1983, Immunology Today, 4: 72; Cole et al., 1983, Proc. Natl. Acad. Sci USA, 80: 2026), and the EBV-hybridoma technique (Cole et al., “Monoclonal Antibodies and Cancer Therapy,” Alan R. Liss, Inc., pp. 77-96 (1983). Such antibodies can be of any immunoglobulin class including IgG, IgM, IgE, IgA, IgD, and any subclass thereof. A hybridoma producing monoclonal antibodies can be cultivated in vitro and in vivo.

Antibody fragments that have specific binding affinity for a vimentin polypeptide can be generated by known techniques. For example, such fragments include but are not limited to F(ab′)2 fragments that can be produced by pepsin digestion of the antibody molecule, and Fab fragments that can be generated by reducing the disulfide bridges of F(ab′)2 fragments. Alternatively, Fab expression libraries can be constructed. See, for example, Huse et al., 1989, Science, 246: 1275. Once produced, antibodies or fragments thereof are tested for recognition of vimentin by standard immunoassay methods including ELISA techniques, radioimmunoassays and Western blotting. See, Short Protocols in Molecular Biology, Chapter 11, Green Publishing Associates and John Wiley & Sons, Edited by Ausubel, F. M. et al., 1992.

Non-Human Mammals

The invention features non-human mammals that contain nucleic acids of the invention, as well as progeny and cells of such non-human mammals. In some cases, the invention features non-human mammals that contain a vimentin nucleic acid of the invention. In other cases, the invention features non-human mammals that contain a vimentin nucleic acid and another nucleic acid of the invention, such as CD151. Non-human mammals include, for example, aquatic animals (e.g., fish, sharks, dolphin, shrimp, and the like), farm animals (e.g., pigs, goats, sheep, cows, horses, rabbits, and the like), rodents (e.g., rats, guinea pigs, and mice), non-human primates (e.g., baboon, monkeys, and chimpanzees), and domestic animals (e.g., dogs and cats). Non-human mammals of the invention can express a vimentin nucleotide sequence in addition to an endogenous vimentin nucleic acid (e.g., a transgenic non-human mammal that includes a vimentin nucleic acid molecule integrated into its genome). In certain instances, non-human mammals of the invention contain a vector that expresses porcine vimentin (such as, for example, the one set forth in SEQ ID NO:1), where the vector includes a CMV promoter and a G418 antibiotic resistance gene. In other instances, non-human mammals of the invention contain a vector that expresses a simian vimentin (such as, for example, the one set forth in SEQ ID NO:3), where the vector includes a SV40 promoter and a G418 antibiotic resistance gene.

Several techniques known in the art can be used to introduce a nucleic acid construct into animals to produce the founder lines of transgenic animals. Such techniques include, without limitation, pronuclear microinjection (U.S. Pat. No. 4,873,191); retrovirus mediated gene transfer into germ lines (Van der Putten et al., Proc. Natl. Acad. Sci., USA, 82:6148 (1985)); gene transfection into embryonic stem cells (Gossler et al., Proc Natl Acad Sci USA 83:9065-9069 (1986)); gene targeting into embryonic stem cells (Thompson et al., Cell, 56:313 (1989)); nuclear transfer of somatic nuclei (Schnieke et al., Science 278:2130-2133 (1997)); and electroporation of embryos (Lo, Mol. Cell. Biol., 3:1803-1814 (1983)). Once obtained, transgenic animals having the desired characteristics can be replicated using traditional breeding or animal cloning.

Various methods can be used to identify a host cell containing a nucleic acid construct provided herein. Such methods include, without limitation, PCR, nucleic acid hybridization techniques such as Northern and Southern analyses, and in situ nucleic acid hybridization. In some cases, immunohistochemistry and biochemical techniques can be used to determine if a cell contains a nucleic acid vector with a particular insert sequence by detecting the expression of a polypeptide encoded by that particular insert sequence.

Screening of Test Agents

Non-human mammals of the invention, such as mice and pigs, could be used to screen, for example, for drugs that alter vimentin polypeptide activity or inhibit PRRSV replication. For example, vimentin polypeptide activity can be assessed in a first group of such non-human mammals in the presence of a particular compound, and compared with vimentin polypeptides activity in a corresponding control group in the absence of the compound. As used herein, suitable compounds include biological macromolecules such as an oligonucleotide (RNA or DNA) or a polypeptide of any length, a chemical compound, a mixture of chemical compounds, or an extract isolated from bacterial, plant, fungal, or animal matter. The concentration of compound to be tested depends on the type of compound and in vitro test data. For example, pigs could used to identify compounds capable of preventing or reducing the development of the disease caused by PRRSV. In some instances, a pig that expresses a vimentin polypeptide could be used to test small molecule chemical libraries for PRRSV treatment compounds. Such a pig can express a vimentin polypeptide (e.g., with the sequence set forth in SEQ ID NO:2), where the vector directing vimentin expression includes a CMV promoter.

Transgenic non-human animals (e.g., a transgenic non-human mammal) can be exposed to test compounds by any route of administration, including enterally (e.g., orally) and parenterally (e.g., subcutaneously, intravascularly, intramuscularly, or intranasally). Suitable formulations for oral administration can include tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g. magnesium stearate, talc or silica); disintegrants (e.g., potato starch or sodium starch glycolate); or wetting agents (e.g., sodium lauryl sulfate). Tablets can be coated by methods known in the art. Preparations for oral administration can also be formulated to give controlled release of the compound. Enteral routes include sublingual and oral administration. Compounds can be prepared for parenteral administration in the form of liquid solutions or suspensions, or for intranasal administration in the form of powders, nasal drops, or aerosols. Administration of compounds may also be topical and/or localized, in the form of salves, pastes, gels, solutions, powders and the like. Compounds can be prepared for other routes of administration using standard techniques. Test compounds can be mixed with non-toxic excipients or carriers before administration. Inhalation formulations can include aqueous solutions containing, for example, polyoxyethylene-9-lauryl ether, glycocholate, or deoxycholate. Other formulations may contain sterile water or saline, or polyalkylene glycols such as polyethylene glycol.

Compounds can be prepared for parenteral administration in liquid form (e.g., solutions, solvents, suspensions, and emulsions) including sterile aqueous or non-aqueous carriers. Aqueous carriers include, without limitation, water, alcohol, saline, and buffered solutions. Examples of non-aqueous carriers include, without limitation, propylene glycol, polyethylene glycol, vegetable oils, and injectable organic esters. Preservatives and other additives such as, for example, antimicrobials, anti-oxidants, chelating agents, inert gases, and the like may also be present. Pharmaceutically acceptable carriers for intravenous administration include solutions containing pharmaceutically acceptable salts or sugars. Intranasal preparations can be presented in a liquid form (e.g., nasal drops or aerosols) or as a dry product (e.g., a powder). Both liquid and dry nasal preparations can be administered using a suitable inhalation device. Nebulised aqueous suspensions or solutions can also be prepared with or without a suitable pH and/or tonicity adjustment.

Vaccines

It is contemplated that a vaccine for use in the prevention, protection and treatment of pigs can be made from a PRRS virus preparation. A vaccine can include an immunogenic amount of an inactivated, attenuated, or live PRRS virus preparation made by propagating PRRSV in a non-simian mammalian host cell as described herein (e.g., a host cell containing a nucleic acid construct that includes a nucleic acid having at least 90% sequence identity to the sequence set forth in SEQ ID NO:1 or SEQ ID NO:3). For example, a PRRSV preparation can be made by infecting a host cell culture with PRRSV (e.g., the Lelystad strain or VR 2332 strain of PRRSV) and incubating the culture until at least 50% (e.g., 55%, 60%, 70%, 75%, 80%, 85%, 90%, or 95%) of the cells exhibit one or more cytopathic effects (e.g., rounded cells, detached cells, or syncytium formation). Typically, the culture is incubated at 34 to 37° C. After one or more cytopathic effects are observed in at least 50% of the cells, the cells can be lysed (e.g., by one or more freeze thaw cycles) and the virus harvested to produce a virus preparation. The virus can be purified after removing cellular debris using, for example, polyethylene glycol precipitation and sucrose density gradient centrifugation. The purified virus can be inactivated to produce a killed virus preparation.

To prepare a vaccine, the virus preparation can be conjugated or linked to a peptide or to a polysaccharide. For example, immunogenic proteins well known in the art, also known as “carriers,” may be employed. Useful immunogenic proteins include keyhole limpet hemocyanin (KLH), bovine serum albumin (BSA), ovalbumin, human serum albumin, human gamma globulin, chicken immunoglobulin G and bovine gamma globulin. Useful immunogenic polysaccharides include group A Streptococcal polysaccharide, C-polysaccharide from group B Streptococci, or the capsular polysaccharides of Streptococcus pnuemoniae or group B Streptococci. Alternatively, polysaccharides or proteins of other pathogens can be conjugated to, linked to, or mixed with the virus preparation.

In some embodiments, the infected cells can be isolated, lyophilized and stabilized. Cells may then be adjusted to an appropriate concentration, optionally combined with a suitable vaccine adjuvant, and packaged for use. Suitable adjuvants include but are not limited to surfactants, e.g., hexadecylamine, octadecylamine, lysolecithin, dimethyldioctadecylammonium bromide, N,N-dioctadecyl-N′-N-bis(2-hydroxyethyl-propane di-amine), methoxyhexadecyl-glycerol, and pluronic polyols; polyanions, e.g., pyran, dextran sulfate, poly IC, polyacrylic acid, carbopol; peptides, e.g., muramyl dipeptide, MPL, aimethylglycine, tuftsin, oil emulsions, alum, and mixtures thereof. Other potential adjuvants include the B peptide subunits of E. coli heat labile toxin or of the cholera toxin. McGhee, J. R., et al., “On vaccine development,” Sem. Hematol., 30:3-15 (1993). Finally, the immunogenic product may be incorporated into liposomes for use in a vaccine formulation, or may be conjugated to proteins such as KLH or HSA or other polymers.

A vaccine typically is administered to a pig parenterally, usually by intramuscular or subcutaneous injection in an appropriate vehicle. Other modes of administration, such as oral delivery or intranasal delivery, are also acceptable. Vaccine formulations typically contain an effective amount of a PRRS virus preparation in a vehicle. The effective amount is sufficient to prevent, ameliorate or reduce the incidence of PRRSV in the target non-human mammal, as determined by one skilled in the art. The active ingredient may range from about 1% to about 95% (w/w) of the composition. The quantity to be administered depends upon factors such as the age, sex, weight and physical condition of the pig considered for vaccination. The quantity also depends upon the capacity of the mammal's immune system to synthesize antibodies, and the degree of protection desired. Effective dosages can be established by one of ordinary skill in the art through routine trials establishing dose response curves. The pig can be immunized by administration of the vaccine in one or more doses. Multiple doses may be administered if required to maintain a state of immunity to PRRSV.

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservatives.

In some cases, pigs can be administered compositions comprising a therapeutically effective amount of a polypeptide and/or nucleic acid, such as the soluble form of a polypeptide and/or nucleic acid, agonist or antagonist peptide or small molecule compound, in combination with an acceptable carrier or excipient. In some instances, pigs can be administered compositions comprising a therapeutically effective amount of a soluble form of vimentin, in combination with dextrose as a carrier. In other instances, pigs can be administered compositions comprising a therapeutically effective amount of a soluble form of vimentin in combination with dextrose as a carrier.

Anti-Vimentin Antibodies

It is contemplated that anti-vimentin antibodies can be used to treat pigs against PRRSV. A composition suitable for administering to a pig can be an ointment, a nebulizable liquid, a powder, or a sprayable liquid that comprises anti-vimentin antibodies. Such antibodies can be produced in a non-porcine species (heterologous antibodies), or can be produced in pigs (homologous antibodies). Thus, a suitable antibody can anti-porcine, anti-simian, or anti-human vimentin antibody. A suitable heterologous antibody is the 7G10 monoclonal antibody described in Shanmukhappa et al., 2000, Hybridoma, 19: 263-267.

Such a composition can be administered to pigs by a number of routes, including, oral, cutaneous, nasal, intraperitoneal, intravaginal, mucosal, or parenteral (i.e., intravenous or intramuscular) administration. Typically, a PRRSV antibody is administered intranasally as a powder or nebulizable liquid in swine houses in the face of an exposure. Administration of antibodies is carried out in an immunologically effective amount for a given infection, disease, or other conditions (e.g., a PRRSV outbreak). Administration of antibodies can also carried out as a preventive measure (e.g., for the prevention of the disease caused by PRRSV). Typical passive immunization doses are in the range of about 0.2 mg of biological per kg of body weight to about 10.0 mg of biological per kg of body weight, e.g., 0.25 mg/kg to 10.0 mg/kg, or 0.4 mg/kg to 9.0 mg/kg, or 0.5 mg/kg to 5.0 mg/kg, or 0.7 mg/kg to 10.0 mg/kg, or 0.75 mg/kg to 5.0 mg/kg, or 0.9 mg/kg to 3.0 mg/kg, or 1.0 mg/kg to 3.0 mg/kg, or 2.5 mg/kg to 5.0 mg/kg, or 4.2 mg/kg to 10.0 mg/kg.

The composition can be administered to a tissue or organ such as the nasal passages, the throat, or the lungs. If the pig is a female, the composition can be administered to the vagina, e.g., as an ointment.

In some embodiments, the composition is mixed with semen. A semen/anti-vimentin antibody composition can be stored until needed for use in an artificial insemination program.

Protein-Protein Interactions

Molecular interactions between a host and a virus are integral to the process of viral infection and disease. Many of these interactions are in the form of protein-protein contacts. Detecting and determining interacting partners can aid in devising methods to disrupt such interactions, and thereby to interrupt a viral infection cycle. Methods to detect protein interactions are known in the art. Such methods include, without limitation, immunoprecipitation with an antibody that binds to the protein in a complex followed by analysis by size fractionation of the immunoprecipitated proteins (e.g. by denaturing or nondenaturing polyacrylamide gel electrophoresis), Western analysis, non-denaturing gel electrophoresis, yeast hybrid assays (e.g., yeast two hybrid assays), and protein-affinity chromatography coupled with MALDI-TOF mass spectrometry (see, e.g., U.S. Pat. No. 6,797,523).

Administration of agents, such as drugs, can disrupt protein-protein interactions. For example, agents that disrupt an interaction between a major host receptor protein and a viral protein can be administered to pigs that were exposed to PRRSV in order to prevent or treat disease development.

Agents that disrupt protein-protein interactions can be identified by methods known to those of skill in the art. These methods include, without limitation, fluorescence assays, such as fluorescence polarization or fluorescence resonance energy transfer (FRET)-based assays, and surface plasmon resonance-based assays. In other cases, determining whether a protein complex is disrupted can be determined, for example, by mobility shift assays. In certain instances, an agent can be screened to determine whether it is useful for preventing or treating a PRRSV infection by contacting a complex of GP5 and vimentin with a test agent, and determining whether this agent disrupts the formation of the complex.

For example, a PRRSV infection can be treated or prevented in a pig by, for example, administering a compound that prevents or inhibits the association of viral GP4 or GP5 with host vimentin. GP4 and GP5 proteins of PRRSV are viral surface glycoproteins (Pirzadeh and Dea, 1998, J Gen Virol., 79: 989-999). GP4 and GP5 possess antigenic determinants (Gagnon et al., 2000, Arch. Virol, 145: 659-688). GP4 alone, or in cooperation with GP5 may interact with host receptor molecules, such as sialoadhesin and/or vimentin. Such interaction is detected as follows. Biotinylated GP4 and GP5, alone or in combination are used to screen mammalian cDNA expression libraries of the CL-2621, MARC-145, or porcine macrophage cell lines, thereby identifying vimentin, sialoadhesin, and other possible PRRSV receptor components. This approach obviates the need for using purified PRRSV and it is easier to use purified GP4 and GP5.

The invention will be further described in the following examples, which do not limit the scope of the invention described in the claims.

### EXAMPLES

### Example 1

### 7G10 Monoclonal Antibody Immunoaffinity Chromatography

A preparation of the monoclonal antibody 7G10 was coupled to IgA-mouse specific beads. A total cell lysate from an uninfected MARC-145 cell line was passed over a column containing the 7G10-linked beads. Unbound proteins were eluted from the column, and bound proteins were eluted as described in Fahad, 2002, Kansas State University, Ph.D. dissertation. Bound proteins were separated by 2D SDS-PAGE and, following silver staining, spots were cored and submitted for MALDI-TOF analysis.

In-gel tryptic digestion, matrix-assisted laser desorption ionization time-of-flight (MALDI-TOF) mass spectrometry, and analysis of peptide sequences were next performed, in order to determine the identity of the host proteins that bound to the 7G10 antibody. In-gel tryptic digestion and MALDI-TOF MS were performed in the proteomics lab at the University of Louisville, Louisville, Ky. Protein identification of peptide fragments was performed by using the “Profound” search engine (available at //129.85.19.192/profound_bin/WebPro Found.exe) based on the entire NCBI protein database using the assumption that peptides are monoisotopic, oxidized at methionine residues and carboxamidomethylated at cysteine residues. Up to one missed trypsin cleavage was allowed, although most matches did not contain any missed cleavages. Mass tolerance of 150 ppm was the window of error to be allowed for matching the peptide mass values. Probability-based MOWSE scores were estimated by comparison of search results against estimated random match population and were reported as 10*log₁₀(P), where P is the absolute probability. Scores greater than 70 were considered significant (P<0.05). All of the proteins that were identified were in the expected range of size and isoelectric point (pI), based on their respective positions in the gels.

The results of the experiment indicated that vimentin, cytokeratin 8, keratin 18, actin, hair type II basic keratin, and two unidentified proteins were bound by the 7G10 antibody. The results suggest that these polypeptides may be components of a PRRSV receptor complex.

### Example 2

### Virus Overlay Protein Binding Assay (VOPBA)

To determine whether purified PRRSV can bind to vimentin, a VOPBA was performed. First, MARC-145 cells were used to propagate PRRSV, by infecting confluent monolayers of MARC-145 cells with PRRS virus strain ATCC VR 2332. Infected MARC-145 cells were harvested when 75% or more of them showed cytopathic effects, such as rounding, detached, and syncytium formation. After three freeze-thaw cycles, the cells were scraped and collected. Cellular debris was removed by centrifugation at 3,500×g for 20 min, and the supernatant was filtered through a 0.45 μm-pore-size filter. Polyethylene glycol 8000 (PEC-8000) (Sigma Chemical Co., St. Louis, Mo.) was added to achieve a final concentration of 8% (wt/vol). Following an overnight or longer incubation at 4° C., supernatant was centrifuged at 10,800×g for 20 min to precipitate the virus. The virus pellet was resuspended in TNE buffer (50 mM Tris-HCl, 100 mM NaCl, 1 mM EDTA, pH 7.5). The resuspended virus pellet was layered on 10 to 60% (wt/wt) sucrose gradient, and centrifuged at 90,000×g at 4° C. overnight. The purified virus band was collected, and its density (g/cm³) was determined to be 1.1513. Centrifugation was then performed at 90,000×g for 1 hr to pellet the purified virus. Purified virus was stored at −70° C.

A vimentin preparation (Fisher Scientific, Cat# BP2545-100, Fair Lawn, N.J.) was subjected to separation by 10% SDS-PAGE at 80 V for 2 hr. The protein was then transferred onto a nitrocellulose membrane (Gelman Sciences, Ann Arbor, Mich.) using a Bio-Rad transfer apparatus at 100 V for 1 hour. The nitrocellulose membrane was soaked in a blocking buffer (10% skim milk in PBS) at 4° C. for overnight. The membrane was next rinsed with PBS three times, each for 10 minutes at room temperature (RT), and incubated with 10 μg of purified virus at 4° C. for overnight. The membrane was washed with PBS three times, each for 10 minutes at RT. The membrane was then incubated with a monoclonal antibody against PRRSV-nucleocapsid protein (SR-30), and a polyclonal antibody against PRRSV, at RT for 1 hour. The membrane was washed with PBS three times, each for 10 minutes at RT, and incubated with secondary antibodies conjugated with peroxidase (horse anti-mouse IgG (H+ L)) (Vector Laboratories, Inc., Burlingame, Calif.) for SR-30, and goat anti-porcine IgG (H+ L) (ICN Biomedicals, Inc., Aurora, Ohio) for the PRRSV-polyclonal antibody, at RT for 2 hours. The membrane was washed with PBS three times, each for 10 min at RT. The presence of antibody-peroxidase conjugate was detected by the addition of substrate, TMB Membrane Peroxidase Substrate (KPL, Gaithersburg, Md.). The results using the SR-30 antibody indicated that PRRSV bound to vimentin.

### Example 3

### Identification of Isoforms Of PRRSV Receptor Components

Isoforms of vimentin were identified by Western blot analysis, using a monoclonal antibody to vimentin. Total cellular proteins were harvested from uninfected MARC-145, BHK-21, CRFK, MDCK, PK-15, ST and Vero cell lines. The proteins were separated by 10% SDS-PAGE at 80 V for 2 hours and transferred to a nitrocellulose membrane (Gelman Sciences, Ann Arbor, Mich.) using a Bio-Rad transfer apparatus at 100 V for 1 hour. The nitrocellulose membrane was soaked in a blocking buffer (5% skim milk in 0.05% Tween 20/PBS) at 4° C. for overnight. The membrane was incubated with an anti-vimentin monoclonal antibody (clone V9) (Sigma-Aldrich Co., St. Louis, Mo.) at RT for 2 hours. The membrane was washed with 0.05% Tween 20/PBS three times, each for 10 minutes at RT, and incubated with a secondary antibody conjugated with peroxidase (horse anti-mouse IgG (H+L) (Vector Laboratories, Inc., Burlingame, Calif.) at RT for 2 hours. The membrane was then washed with 0.05% Tween 20/PBS three times, each for 10 minutes at RT. The expression of vimentin was detected by the addition of substrate, TMB Membrane Peroxidase Substrate (1 component, KPL, Gaithersburg, Md.). The molecular mass of vimentin differed among the cell lines tested. In addition, multiple bands were observed in the cell lines, possibly due to different degrees of phosphorylation.

### Example 4

### Isolation and Sequencing of Vimentin Nucleic Acids

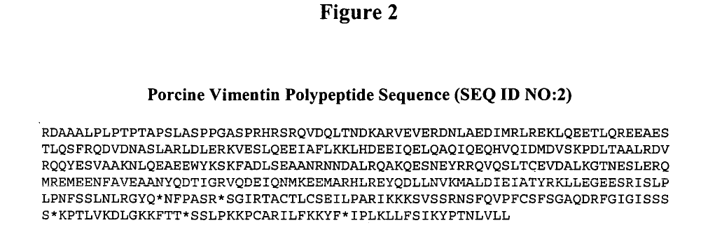

Total porcine RNA was extracted from a swine testicle cell line. Vimentin mRNA was amplified by RT-PCR, using forward (5′-AGAGACGCAGCAGCGCTC CCTT-3′) (SEQ ID NO:11) and reverse (5′-GAAGCAGAACCAAGTTGGTTGG-3′) (SEQ ID NO:12) primers, which were synthesized based on the human vimentin sequence. The reverse transcription reaction conditions were as follows: 42° C. for 45 minutes, 95° C. for 10 minutes, and 5° C. for 5 minutes, in the presence of 2.5 nM MgCl₂ and no DMSO. Amplification was carried out using the following PCR steps: (1) 95° C. for 2 minutes, (2) 95° C. for 30 seconds, (3) 53° C. for 90 seconds, (4) 72° C. for 90 seconds, (5) repeat sequential steps (2)-(4) 24 times, and (6) finalize extensions at 72° C. for 30 minutes. The resulting amplification product of approximately 1813 bp was cloned in a pGEM-T vector essentially according to the manufacturer's instructions (Promega, Madison, Wis.). The amplification product was sequenced at a DNA Sequencing Laboratory at Iowa State University (Ames, Iowa). The nucleotide sequence is shown in FIG. 1. The amino acid sequence of the corresponding polypeptide is shown in FIG. 2.

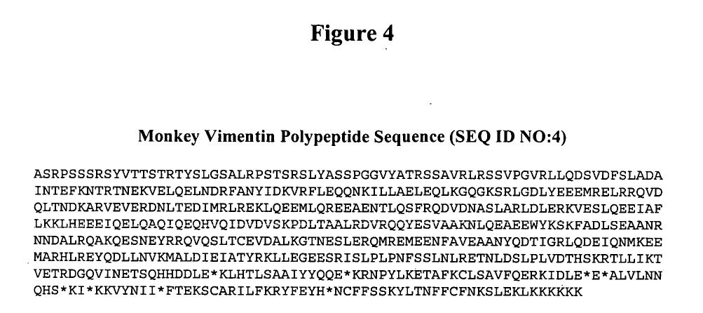

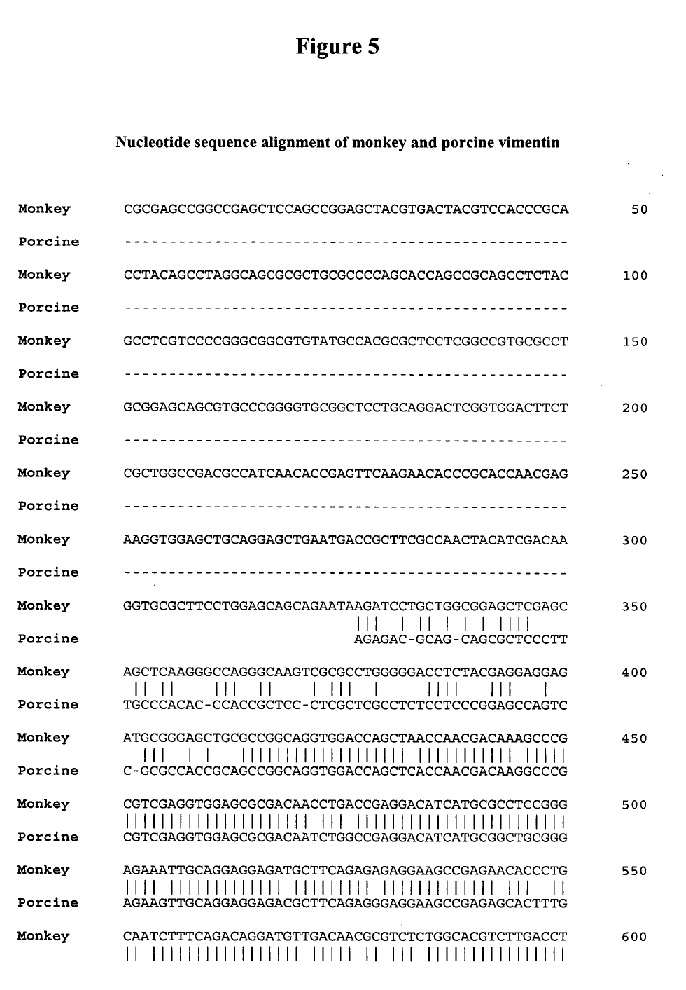

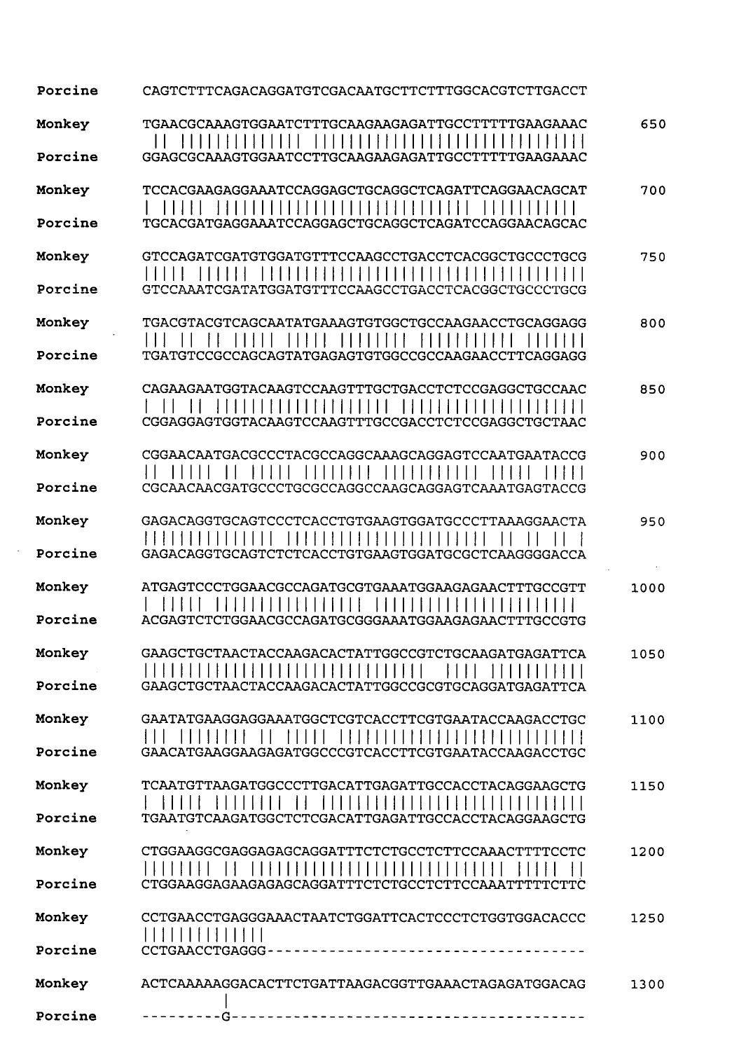

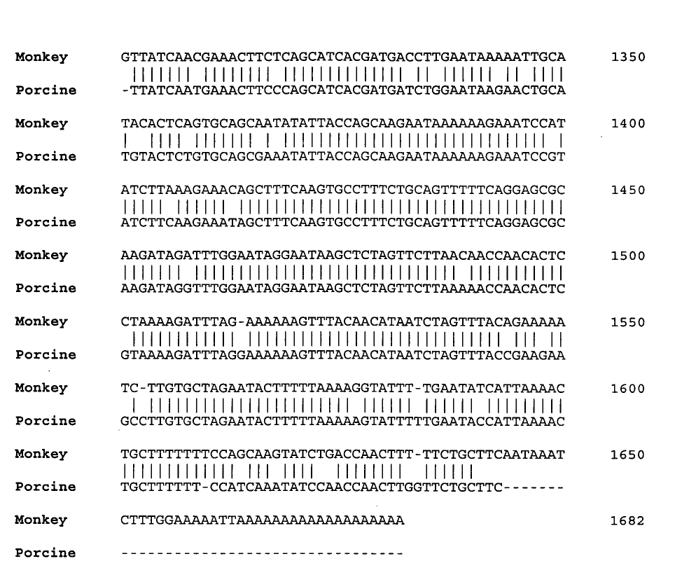

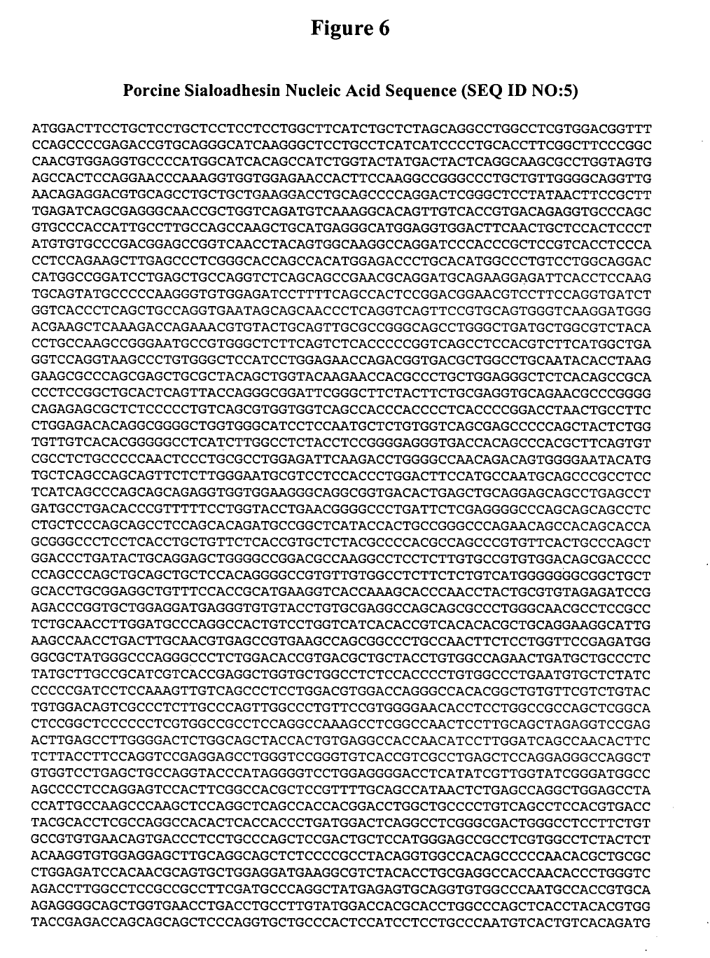

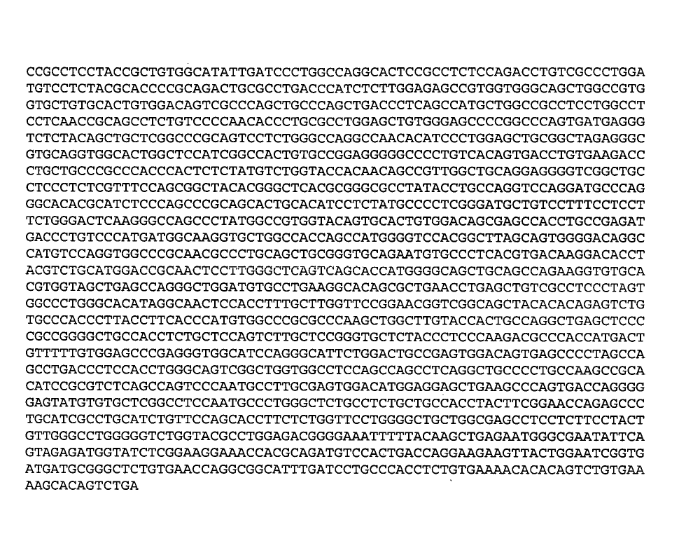

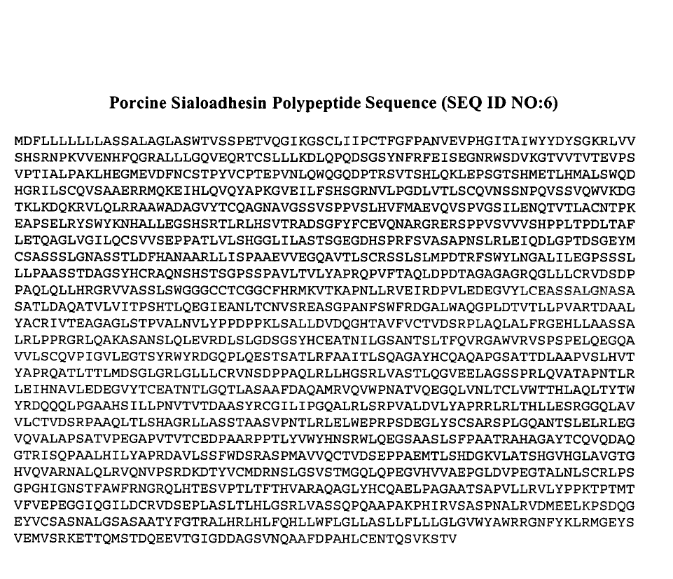

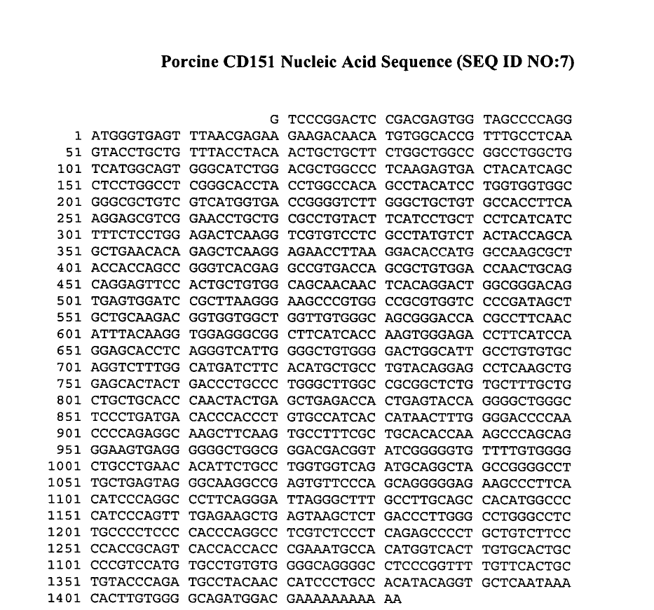

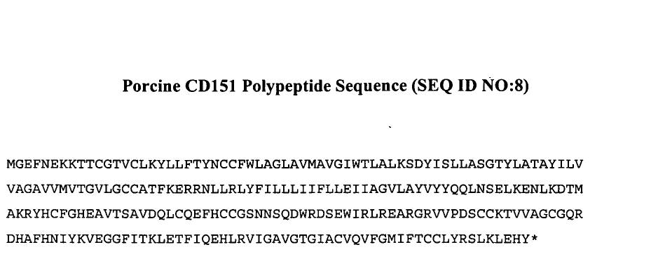

Simian vimentin cDNA was isolated from the MARC-145 cell line by screening a MARC-145 expression library with porcine vimentin cDNA, and the sequence of one of the hybridizing colonies was determined. The simian vimentin nucleotide sequence is shown in FIG. 3. The amino acid sequence of the corresponding polypeptide is shown in FIG. 4.

### Example 5

### Receptor Blocking By Polyclonal Antivimentin Antibody

In order to determine whether occupation of the vimentin receptor domain affects the binding of PRRSV to vimentin, a blocking test was performed. A polyclonal antibody (rabbit anti-human vimentin Cat# V2009, Biomeda, Foster City, Calif.) was used in a checker board titration of polyclonal antibody and PRRSV (strain ATCC VR 2332). The PRRSV preparation was 1:10-serially diluted from 1:10. The anti-vimentin antibody was 1:2-serially diluted from 1:20. The intensity of the indirect fluorescence and the number of fluorescence-positive cells were determined and used to semiquantitatively estimate the amount of PRRSV present in each well. The results showed that the antibody was cytotoxic at dilutions of 1:20, 1:40 and 1:80. No PRRS virus was detected in the presence of antibody at antibody dilutions of 1:160 to 1:5, 120. However, PRRSV was detected in the absence of antibody at virus dilutions up to 10⁻⁷. The results showed that an anti-vimentin antibody is able to block PRRSV propagation in a dose dependent manner.

### Example 6

### Detection of Interaction Between PRRSV Surface Proteins and Host Receptor Components

A purified preparation of PRRSV GP4 is biotinylated. The biotinylated GP4 preparation is used to screen a cDNA expression library from MARC-145 cells for polypeptides that bind to GP4. MARC-145 polypeptides that bind to GP4 are sequenced and characterized. The experiment is repeated with cDNA expression libraries from CL-2621 cells and porcine macrophage cells.

A biotinylated preparation of PRRSV GP5 is used to screen a cDNA expression library from MARC-145 cells for polypeptides that bind to GP5. MARC-145 polypeptides that bind to GP5 are sequenced and characterized. The experiment is repeated with cDNA expression libraries from CL-2621 cells and porcine macrophage cells.

A biotinylated preparation of PRRSV GP4 and GP5 is used to screen a cDNA expression library from MARC-145 cells for polypeptides that bind to both GP4 and GP5. MARC-145 polypeptides that bind to both GP4 and GP5 are sequenced and characterized. The experiment is repeated with cDNA expression libraries from CL-2621 cells and porcine macrophage cells.

### Example 7

### Cell Line Susceptible to PRRSV and Use in Swine Vaccination

Louise C. Averill Laboratory non-simian cell lines are infected with an American PRRSV isolate. Infected cells are observed for cytopathic effects and are monitored for high PRRSV titers. Infected cells having high PRRSV titers are lysed and mixed with an adjuvant. The mixture is administered intramuscularly to pigs. Humoral and cellular-mediated immunity (CMI) responses are determined for each animal, as well as the ability of each animal to withstand a challenge PRRSV viral infection. The experiment is repeated using a European PRRSV isolate or an atypical PRRSV isolate.

### Example 8

### Vimentin Polymorphisms and Swine Breeding

Chromosomal DNA is extracted from ear notch biopsies of Yorkshire, Duroc, and Poland-China pigs. Vimentin genomic DNA is amplified by PCR and single nucleotide polymorphisms or RFLPs are identified. Statistical analysis is performed to identify any correlations between pig breed and susceptibility to PRRSV. The experiment is repeated using Chester White, Berkshire and Hampshire pigs. Pigs that demonstrate vimentin polymorphism(s) that correlate with PRRS resistance are selected for further breeding.

### Example 9

### Localization of Vimentin in MARC-145 Cells

An immunofluorescence assay was used to determine the sub-cellular localization of vimentin in MARC-145 cells. MARC-145 cells (5×10⁵ cells) permeabilized in 80% acetone were washed in staining solution (0.1% BSA and 0.1% NaN₃ in PBS), resuspended in 50 μl of blocking solution (3% normal goat serum in staining solution), then incubated for 10 mins on ice. After blocking, the cells were incubated with polyclonal rabbit anti-vimentin Ab (Biomeda, Foster City, Calif., USA) for 30 mins on ice at a final concentration of 1:100-dilution. After washing twice with staining solution, the cells were incubated with 1 μg of FITC-conjugated goat anti-rabbit IgG (H+L) (BethyL lavortories, Inc., Montgomery, Tex., USA) for 30 mins on ice. After washing twice with staining solution, the cells were resuspended in 300 μl of 1% paraformaldehyde in PBS, and then flow cytometric analysis was performed on a FACSCalibur (BD Biosciences, San Jose, Calif., USA). Dead cells were gated out by their low forward angle light scatter intensity. Ten thousand cells were scored per measurement. Vimentin was found in the cytoplasm and was especially concentrated around the plasma membrane.

To examine the cell surface expression of vimentin, a similar assay was performed on non-permeabilized MARC-145 cells. Staining was detected on the surface of MARC-145 cells. Furthermore, the expression of vimentin on the cell surface was changed by PRRSV infection. Up to 2 days after post-infection of PRRSV, the expression level of vimentin was reduced to half of uninfected cells. However, the expression level of vimentin was restored to the same level as uninfected cells at 3 days after post-infection.

### Example 10

### Rendering Non-Susceptible Cells Susceptible to PRRSV

Simian recombinant protein was produced in the QIAexpressionist™ system (Qiagen, Valencia, Calif., USA). The full length simian vimentin cDNA (SEQ ID NO:3) was digested with Kpn I and Bam HI, and ligated to a Kpn I and Bam HI-digested pQE-30 vector. The ligation-reaction mixture was transformed into competent M15 E. coli. After inducing the expression of recombinant vimentin by IPTG at a final concentration of 1 mM, recombinant vimentin was purified by Ni-NTA column.

Recombinant vimentin was transfected into BHK-21 cells using the Chariot™ protein delivery system (Active Motif, Inc., Carlsbad, Calif., USA) according to the manufacturer's instructions. In brief, the Chariot™ reagent and vimentin were incubated at room temperature for 30 minutes to form a Chariot™-vimentin complex. BHK-21 cells, which had been cultured overnight in a 48-well tissue culture plate (5×10⁴ cells/well), were prepared for the Chariot™-vimentin complex by removing the media and washing the cells twice with PBS. After adding 100 μL of the Chariot™-vimentin complex and 100 μl of serum-free media to each well, the cells were incubated at 37° C. for 1 hr. Three hundred microliters of complete growth media then were added into each well, and the cells were incubated at 37° C. for 2 hrs.

After incubating the transfected cells for 3 days with PRRSV, the cells were stained with FITC-conjugated SDOW-17, a monoclonal antibody against PRRSV-nucleocapsid protein, and examined by fluorescence microscopy for PRRSV. The simian vimentin-transfected BHK-21 cells were rendered susceptible to PRRSV.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A non-simian, mammalian cell whose genome comprises a nucleic acid construct, said nucleic acid construct comprising a nucleic acid having at least about 90% sequence identity to the sequence set forth in SEQ ID NO:1 or SEQ ID NO:3.

2. The cell of claim 1, wherein said nucleic acid has the sequence set forth in SEQ ID NO: 1.

3. The cell of claim 1, wherein said nucleic acid has the sequence set forth in SEQ ID NO:3.

4. The cell of claim 1, said nucleic acid construct further comprising a nucleic acid having at least about 90% sequence identity to the sequence set forth in SEQ ID NO:7.

5. The cell of any of claims 1 through 4, wherein said cell is a swine testicular cell line.

6. The cell of any of claims 1 through 4, wherein said cell is a primary swine kidney cell line.

7. A method for making a virus preparation, comprising:
a) infecting a culture of the cell of claim 1 with PRRS virus,
b) incubating said culture until at least 50% of the cells exhibit one or more cytopathic effects;
c) lysing the cells; and
d) harvesting PRRS virus from the lysed cells to produce said virus preparation.

8. The method of claim 7, further comprising the step of purifying PRRS virus, after said harvesting step, to produce said virus preparation.

9. The method of claim 7, wherein said virus is the Lelystad strain of PRRSV.

10. The method of claim 7, wherein said virus is the VR 2332 strain of PRRSV.

11. The method of claim 7, further comprising the step of inactivating said virus, after said harvesting step, to produce said virus preparation.

12. The method of claim 7, wherein said cell is a swine testicular cell line or a primary swine kidney cell line.

13. The method of claim 7, wherein said nucleic acid construct comprises a nucleic acid having the sequence set forth in SEQ ID NO: 1 or SEQ ID NO:3.

14. A vaccine made from PRRS virus, said virus prepared by the method of any one of claims 7 through 13.

15. A method of treating a PRRSV infection, comprising the step of administering to a pig a composition comprising a virus preparation made by the method of any of claims 7 through 13.

16. A method for treating PRRSV infection, said method comprising administering a composition comprising an anti-vimentin antibody to a pig.

17. The method of claim 16, wherein said composition is selected from the group consisting of an ointment, a nebulizable liquid, a powder, and a sprayable liquid.

18. The method of claim 16, wherein said anti-vimentin antibody is an anti-porcine vimentin antibody.

19. The method of claim 18, wherein said anti-vimentin antibody is 7G10 monoclonal antibody.

20. The method of any of claims 16 through 19, wherein said pig is a female.

21. The method of claim 20, wherein said composition is administered to a tissue selected from the group consisting of the nasal passages, the throat, the vagina, and the lungs.

22. A method of screening for nucleic acid polymorphisms that are correlated with PRRSV susceptibility, comprising:
a) determining part or all of the vimentin nucleotide sequence in a plurality of pigs; and
b) determining whether any polymorphisms present in said vimentin nucleotide sequence are correlated with PRRSV susceptibility among said pigs.

23. The method of claim 22, wherein said plurality of pigs are of at least two different breeds, each said breed having a different susceptibility to PRRSV relative to at least one other of said breeds

24. An isolated nucleic acid comprising a sequence having about 90% or more sequence identity to SEQ ID NO:1.

25. The isolated nucleic acid of claim 24, wherein said sequence has about 95% or more sequence identity to SEQ ID NO:1.

26. The isolated nucleic acid of claim 24, wherein said sequence is SEQ ID NO:1.

27. An isolated nucleic acid encoding a polypeptide having about 90% or more sequence identity to SEQ ID NO:2.

28. An isolated nucleic acid comprising a sequence having about 90% or more sequence identity to SEQ ID NO:3.

29. An isolated nucleic acid encoding a polypeptide having about 90% or more sequence identity to SEQ ID NO:4.

30. The nucleic acid of any of claim 24 through 29, said nucleic acid further comprising a control element operably linked to said nucleic acid.

31. The nucleic acid of claim 30, wherein said control element is a CMV promoter or an SV40 promoter.

32. An isolated polypeptide having about 90% or more sequence identity to SEQ ID NO:2.

33. The polypeptide of claim 32, wherein said polypeptide has the sequence set forth in SEQ ID NO:2.

34. A method for rendering cells susceptible to PRRSV, said method comprising providing cells resistant to PRRSV and introducing a nucleic acid construct into said cells, wherein said nucleic acid construct comprises a nucleic acid encoding a vimentin polypeptide.

35. The method of claim 34, wherein said nucleic acid has at least about 90% sequence identity to the sequence set forth in SEQ ID NO:1 or SEQ ID NO:3.
